(19) United States
(12) Patent Application Publication
(10) Pub. No.: US 2024/0029834 A1
(43) Pub. Date: Jan. 25, 2024

(54) Drug Optimization by Active Learning

(71) Applicant: EXSCIENTIA AI LIMITED, Dundee (GB)

(72) Inventor: Emil Nicolae NICHITA, Oxford (GB)

(21) Appl. No.: 18/231,219

(22) Filed: Aug. 7, 2023

Related U.S. Application Data
(63) Continuation of application No. PCT/GB2022/050332, filed on Feb. 8, 2022.

(30) Foreign Application Priority Data
Feb. 8, 2021 (GB) GB2101703.3

(51) Int. Cl. G16C 20/50; G16C 20/70; G16C 20/30

(52) U.S. Cl. CPC G16C 20/50; G16C 20/70; G16C 20/30

(57) ABSTRACT

A method for computational drug design by active learning includes defining a population of compounds, defining a training set of compounds from the population for which a plurality of biological properties are known, and defining a plurality of objectives each defining a desired biological property. The method includes training, using the training set, a Bayesian statistical model to output a probability distribution approximating biological properties of compounds in the population as an objective function of structural features of the compounds in the population. The method includes determining, from the population, a subset of compounds that are not in the training set. The subset is determined according to an optimization of an acquisition function based on the probability distribution from the trained Bayesian statistical model and based on the defined objectives. The method includes selecting at least some of the compounds in the determined subset for synthesis.

### RELATED APPLICATIONS

This application is a continuation of PCT Patent Application No. PCT/GB2022/050332, filed on Feb. 8, 2022, entitled “Drug Optimisation by Active Learning,” which claims priority to GB Patent Application No. GB2101703.3, filed on Feb. 8, 2021, entitled “Drug Optimisation by Active Learning,” each of which is incorporated by reference herein in its entirety.

### FIELD OF THE INVENTION

The invention relates to methods and systems for the computational design of compounds, such as drugs. In particular, the invention relates to methods for the optimization of computational models through active learning, to be used in the design of drugs that interact with selected target molecules, and relates to the drugs designed using these systems and methods.

### BACKGROUND

Drug discovery is the process of identifying candidate compounds for progression to the next stage of drug development, such as pre-clinical trials. Such candidate compounds are required to satisfy certain criteria for further development. Modern drug discovery involves the identification and optimization of initial screening ‘hit’ compounds. In particular, such compounds need to be optimized relative to required criteria, which can include the optimization of a number of different biological properties. The properties to be optimized can include, for instance: efficacy/potency against a desired target; selectivity against non-desired targets; low probability of toxicity; and good drug metabolism and pharmacokinetic properties (ADME). Only compounds satisfying the specified requirements become candidate compounds that can continue to the drug development process.

The drug discovery process can involve making/synthesizing a significant number of compounds during the optimization from initial screening hits to candidate compounds. In particular, those compounds that are synthesized are measured to determine their properties, such as biological activity. However, the number of compounds that could be made as part of a particular drug discovery project will far outnumber—likely by orders of magnitude—the number of compounds that can be synthesized and tested. The results of the measurements of synthesized compounds are therefore analyzed and used to inform a decision on which compounds to synthesize next to maximize the likelihood of obtaining compounds with further improved properties relative to the various criteria required by a candidate compound.

The synthesis and subsequent measurement of biological properties, such as biological activity, of one or more compounds at a particular stage is referred to as a design cycle (or iteration) of the drug discovery process. Typically, a set of compounds is synthesized and tested at each design cycle of the process as this is more efficient than synthesizing and testing a single compound at a time. However, a level of available resources usually means that there is an upper limit on the number of compounds in a set that can be synthesized at any given design cycle.

During a wet-lab based drug discovery project, many hundreds or even thousands of compounds typically are synthesized across several design cycles before a candidate compound is found. This is a lengthy, expensive, and inefficient process: synthesis of a single compound can cost thousands of dollars, and it can take three to five years on average to obtain a single candidate compound.

The use of computational methods greatly increases the level of analysis that may be performed on already-synthesized compounds relative to that which could be performed by a medicinal chemist alone. In particular, machine learning (ML), artificial intelligence (AI), or other mathematical methods can be used to evaluate numerous design parameters in parallel, at a level that is beyond the capabilities of a human, to identify relationships between parameters (such as structural features of compounds) and desired properties, such as biological activity levels. The mathematical methods can then use these identified relationships to make a better prediction as to which compounds are more likely to exhibit a greater number/level of desired biological properties relative to required criteria of a candidate compound. This means that such mathematical methods can be used to reduce the number of design cycles, and so reduce the number of compounds that need to be synthesized, to obtain a compound that achieves a desired combination of properties, as required of a candidate compound, thereby achieving an associated reduction in cost and time of a drug discovery project.

The task of finding a candidate compound having a number of desired properties may therefore be regarded as an optimization problem, with the aim of obtaining an ‘optimal’ compound having various desired properties using knowledge obtained from previously-synthesized compounds. There are a number of challenges to be addressed when faced with such a computational optimization problem in the context of drug discovery.

One challenge is that the types of functional relationships between compounds in a population of compounds are not known a priori. That is, the form of an objective function describing relationships between structural features and biological properties of compounds, for instance, is unknown. This means that some known optimization techniques that rely on prior knowledge of the form of a function may not be suitable in the context of drug discovery. Another challenge is that evaluation of the objective function at points of the input space is costly. This is because synthesizing and testing a compound, i.e., the evaluation cost, is both time consuming and expensive. As such, a training set of evaluated points from which the objective function is to be approximated may contain relatively few points, and it is likely not feasible to greatly increase the size of the training set over a short period of time. This can impact how effectively a model approximating the objective function can be trained, and so impact how capable such a model is of making accurate predictions or approximations.

A further challenge is that many known optimization techniques are designed to select a single point at which to evaluate the unknown function. However, as mentioned above, in a drug discovery project it is typically the case that multiple compounds are selected for synthesizing and testing at any given design cycle for reasons of efficiency. That is, multiple points need to be optimized and selected simultaneously for evaluation at a given iteration.

Also, known optimization techniques may be used to optimize a single parameter of an objective function, i.e., the optimization routine has a single objective to optimize against. However, as described above, there will typically be a number of criteria against which a compound needs to be optimized in order to be a suitable candidate compound. That is, multiple parameters of the function need to be optimized in parallel according to various desired biological properties of a candidate compound of the particular drug discovery project under consideration.

Finally, many optimization routines rely on the input space of the objective function being continuous so that techniques such as gradient-based approaches may be used. Clearly, however, in the context of drug discovery the input space is discrete (with each compound representing a point in the input space) and so techniques that rely on a continuous input space may not be utilized.

It is against this background to which the invention is set.

### SUMMARY OF THE INVENTION

According to an aspect of the present invention there is provided a method for computational drug design. The method includes defining a population of a plurality of compounds, each compound having one or more structural features. The method includes defining a training set of compounds from the population for which a plurality of properties are known. The properties may be any relevant physical, chemical or biological property of a compound, which may be considered to encompass biological, biochemical, chemical, biophysical, physiological and/or pharmacological properties of the compounds. The method includes defining a plurality of objectives each defining a desired property. The method includes training, using the training set of compounds, a Bayesian statistical model to output a probability distribution approximating properties of compounds in the population as an objective function of structural features of the compounds in the population. The method includes determining a subset of a plurality of compounds from the population which are not in the training set. The subset is determined according to an optimization of an acquisition function based on the probability distribution from the trained Bayesian statistical model and based on the defined plurality of objectives. The method may include selecting at least some of the compounds in the determined subset for synthesis and/or for performing (computational) molecular dynamics analysis/simulations. This selection may be made as part of a drug design process to obtain a compound with the desired properties. Conveniently, throughout this disclosure such properties of a compound may collectively be referred to as ‘biological properties’ and, therefore, as used herein, a ‘biological property’ may encompass any relevant property of a (chemical) compound, including such properties that might more specifically be considered to fall within the scope of/overlap with biological, biochemical, chemical, biophysical, physiological and/or pharmacological properties.

The method may include, for one or more of the objectives, mapping a preference associated with the biological property of the respective objective by applying a respective utility function to the probability distribution from the Bayesian statistical model to obtain a preference-modified probability distribution. The optimization of the acquisition function may be based on the preference-modified probability distribution.

The preference may be indicative of a priority of the respective objective relative to other ones of the plurality of objectives.

In some embodiments, for one of the biological properties of one of the compounds, it may be the case that a lower uncertainty value associated with the probability distribution for the biological property corresponds to a greater preference associated with the respective biological property.

The preference may be a user-defined preference, for instance by a chemist.

One or more of the utility functions may be piecewise functions. The piecewise functions may be piecewise linear functions.

In some embodiments, optimizing the acquisition function may comprise evaluating the acquisition function for each compound in the population, optionally excluding the compounds in the training set. The subset may be determined based on the evaluated acquisition function values.

In some embodiments, the optimization of the acquisition function based on the defined plurality of objectives may provide a Pareto-optimal set of compounds. One or more of the plurality of compounds for the determined subset may be selected from the Pareto-optimal set. It may be that selection from the Pareto-optimal set is according to user-defined preference.

The probability distribution from the Bayesian statistical model may include a probability distribution for each biological property associated with each respective one of the plurality of objectives.

The method may include mapping the plurality of probability distributions from the Bayesian statistical model to a one-dimensional aggregated probability distribution by applying an aggregation function to the plurality of probability distributions. Optimization of the acquisition function may be based on the aggregated probability distribution.

The aggregation function may comprise one or more of: a sum operator; a mean operator; and a product operator.

The acquisition function may be at least one of: an expected improvement function; a probability of improvement function; and a confidence bounds function.

The acquisition function may be a multi-dimensional acquisition function. In some embodiments, each dimension may correspond to a respective objective of the plurality of objectives. Optionally, the multi-dimensional acquisition function may be a hypervolume expected improvement function.

In some embodiments, training the Bayesian statistical model may include tuning a plurality of hyperparameters of the Bayesian statistical model. Optionally, tuning the hyperparameters may include application of a combination of a maximum likelihood estimation technique and a cross validation technique.

In some embodiments, determining the subset of the plurality of compounds may include identifying one compound from the population that is not in the training set by optimizing the acquisition function based on the probability distribution from the trained Bayesian statistical model and based on the defined plurality of objectives. The method may include repeating the steps of: retraining the Bayesian statistical model using the training set of compounds and the one or more identified compounds; and, identifying one compound from the population that is not in the training set, and which is not the one or more previously identified compounds, by optimizing the acquisition function based on the probability distribution from the retrained Bayesian statistical model and based on the defined plurality of objectives, until the plurality of compounds have been identified for the subset.

In some embodiments, retraining the Bayesian statistical model may include setting one or more fake or dummy biological property values for the one or more identified compounds in the Bayesian statistical model.

The fake biological property values may be set according to one of: a kriging believer approach; and a constant liar approach.

In the Bayesian statistical model, each compound may be represented as a bit vector with the bits indicating the presence or absence of respective structural features in the compound.

The Bayesian statistical model may be a Gaussian process model.

The probability distribution from the trained Bayesian statistical model may include a posterior mean indicative of approximated biological property values of compounds in the population. The probability distribution from the trained Bayesian statistical model may include a posterior variance indicative of an uncertainty associated with the approximated biological property values in the population.

In some embodiments, one or more weighting parameters of the acquisition function may be modified in accordance with a desired strategy of a drug discovery process or project utilizing the described computational drug design method.

The desired strategy may include a balance between an exploitation strategy, dependent on a weighting parameter of the acquisition function associated with the posterior mean, and an exploration strategy, dependent on a weighting parameter of the acquisition function associated with the posterior variance.

The weighting parameters may be user-defined to set the desired strategy.

The Bayesian statistical model may use a kernel indicative of a similarity between pairs of compounds in the population to approximate the biological properties of the compounds.

The kernel may be a Tanimoto similarity kernel.

The method may include synthesizing at least some of the selected compounds of the determined subset to determine biological properties of the selected compounds.

The method may include adding the synthesized compounds to the training set to obtain an updated training set.

The method may include: training, using the updated training set of compounds, an updated Bayesian statistical model to output the probability distribution approximating the objective function; determining a new subset of a plurality of compounds from the population which are not in the updated training set, the new subset being determined according to an optimization of the acquisition function that is dependent on the approximated biological properties from the updated Bayesian statistical model and on the defined plurality of objectives; and, selecting at least some of the compounds in the determined new subset for synthesis.

The method may include synthesizing the selected compounds of the determined new subset to determine biological properties of the selected compounds.

The method may include updating the training set by adding the synthesized compounds thereto.

The method may include iteratively performing the steps of: training, using the updated training set of compounds, an updated Bayesian statistical model to output the probability distribution approximating the objective function; determining a new subset of a plurality of compounds from the population which are not in the updated training set, the new subset being determined according to an optimization of the acquisition function that is dependent on the approximated biological properties from the updated Bayesian statistical model and on the defined plurality of objectives; selecting at least some of the compounds in the determined new subset for synthesis; synthesizing the selected compounds of the determined subset to determine biological properties of the selected compounds; and, adding the synthesized compounds to the training set to obtain an updated training set, until a stop condition is satisfied.

The stop condition may include at least one of: one or more of the synthesized compounds achieve the plurality of objectives; one or more of the synthesized compounds are within acceptable thresholds of the respective plurality of objectives; and a maximum number of iterations have been performed.

In some embodiments, a synthesized compound that achieves the plurality of objectives, or is within acceptable thresholds of the respective plurality of objectives, may be a candidate drug or therapeutic molecule having a desired biological, biochemical, physiological and/or pharmacological activity against a predetermined target molecule.

The predetermined target molecule may be an in vitro and/or in vivo therapeutic, diagnostic, or experimental assay target.

The candidate drug or therapeutic molecule may be for use in medicine; for example, in a method for the treatment of an animal, such as a human or non-human animal.

Each of the objectives may be user-defined, for instance by a chemist defining desired criteria that a candidate compound is to satisfy.

In some embodiments, each of the objectives includes at least one of: a desired value for the respective biological property; a desired range of values for the respective biological properties; and a desired value for the respective biological property to be maximized or minimized.

A number of compounds in the selected subset may be user-defined, for instance based on a level of resources available to test compounds at each design cycle or iteration of a drug design project.

The structural features of each of the plurality of compounds in the population may correspond to fragments present in the compound.

The fragments present in each of the plurality of compounds may be represented as a molecular fingerprint. Optionally, the molecular fingerprint is an Extended Connectivity Fingerprint (ECFP), optionally ECFP0, ECFP2, ECFP4, ECFP6, ECFP8, ECFP10 or ECFP12.

The biological properties may include one or more of: activity; selectivity; toxicity; absorption; distribution; metabolism; and excretion.

According to another aspect of the invention there is provided a compound identified by the method described above.

According to another aspect of the invention there is provided a non-transitory, computer-readable storage medium storing instructions thereon that when executed by a computer processor causes the computer processor to perform the method described above.

According to another aspect of the invention there is provided a computing device for computational drug design. The computing device includes an input arranged to receive data indicative of a population of a plurality of compounds, each compound having one or more structural features. The input is arranged to receive data indicative of a training set of compounds from the population for which a plurality of biological properties are known. The input is arranged to receive data indicative of a plurality of objectives each defining a desired biological property. The computing device includes a processor arranged to train, using the training set of compounds, a Bayesian statistical model to provide a probability distribution approximating biological properties of compounds in the population as an objective function of structural features of the compounds in the population. The processor is arranged to determine a subset of a plurality of compounds from the population which are not in the training set, the subset being determined according to an optimization of an acquisition function based on the probability distribution from the trained Bayesian statistical model and based on the defined plurality of objectives. The computing device includes an output arranged to output the determined subset. Optionally, the computing device is arranged to select at least some of the compounds in the determined subset for synthesis and/or for performing (computational) molecular dynamics analysis/simulations. Alternatively, this may be by user-selection. Optionally, the computing device is arranged to perform said molecular dynamics analysis/simulations.

### BRIEF DESCRIPTION OF THE DRAWINGS

Examples of the invention will now be described with reference to the following drawings, in which:

function.

FIG. 1 illustrates a Gaussian Process model approximation of a defined

FIGS. 2(a), 2(b), 2(c), and 2(d) collectively illustrate how a Gaussian Process model and an acquisition function are used to optimize an objective function as part of an iterative process.

FIG. 3 illustrates an example of a piecewise linear function.

FIG. 4 schematically illustrates application of one or more utility functions and/or aggregation functions to multi-dimensional posterior probability distributions output from a Gaussian Process model trained using a population of compounds.

FIG. 5 shows the steps of a computational drug design method according to an example of the invention.

FIGS. 6(a), 6(b), and 6(c) show plots comparing known and predicted values of biological activities of a test set of molecules. In particular, FIG. 6(a) shows a comparison between the known values and those predicted by the method of FIG. 5. FIG. 6(b) shows a comparison between the known values and those predicted by a prior art method. FIG. 6(c) shows a comparison between the values as predicted by the prior art method and the method of FIG. 5.

FIGS. 7(a) and 7(b) show plots comparing known and predicted values of biological activities of the test set of molecules of FIG. 6, and with a set variance threshold in the method of FIG. 5. In particular, FIG. 7(a) shows a comparison between the known values and those predicted by the method of FIG. 5. FIG. 7(b) shows a comparison between the known values and those predicted by a prior art method.

FIG. 8 shows a plot of how the mean squared error (MSE) and the variance of the method of FIG. 5 varies according to model certainty for the test set of FIG. 6.

FIG. 9 schematically illustrates steps for performing benchmarking of the method of FIG. 5.

FIG. 10(a) shows a plot illustrating a distribution of biological activity values, for a particular activity parameter, of molecules in a test set of molecules, and illustrates a training set of molecules, from the test set, for performing the method of FIG. 5, a selected set of molecules, from the test set, selected by the method of FIG. 5, and a remaining (unknown) set of molecules in the test set not in the training set or selected set. FIG. 10(b) shows a plot illustrating the distribution of biological activity values of molecules in the training set and selected set of FIG. 10(a).

FIG. 11(a) shows a plot illustrating a distribution of biological activity values, for a different activity parameter from FIG. 10(a), of molecules in the test set of molecules of FIG. 10(a). FIG. 11(a) illustrates a training set of molecules, from the test set, for performing the method of FIG. 5, a selected set of molecules, from the test set, selected by the method of FIG. 5, and a remaining set of molecules in the test set not in the training set or selected set. FIG. 11(b) shows a plot illustrating the distribution of biological activity values of molecules in the training set and selected set of FIG. 11(a).

FIG. 12 shows a plot indicating the values of the activity parameters of the molecules in the test set of FIGS. 10(a), 10(b), 11(a), and 11(b), and indicates which molecules are selected by the method of FIG. 5.

FIG. 13 shows a plot illustrating a distribution of relative free binding energy values of molecules in a test set of molecules, and illustrates a training set of molecules, from the test set, for performing the method of FIG. 5, a selected set of molecules, from the test set, selected by the method of FIG. 5, and a remaining (unknown) set of molecules in the test set not in the training set or selected set.

FIG. 14(a) shows a plot of how a cumulative relative free binding energy of a selected set of molecules from the test set of FIG. 13 varies with successive iterations of the method of FIG. 5, compared against optimally selected sets and randomly selected sets. FIG. 14(b) shows a plot of a percentage of the selected molecules in FIG. 14(a) after 30 iterations of the method of FIG. 5 that are in the top x of molecules in the test set according to minimizing the relative free binding energy.

FIG. 15(a) shows the plot of FIG. 14(a), except that FIG. 15(a) shows results of a random forest model greedily selected sets instead of the sets selected via the method of FIG. 5. FIG. 15(b) shows a plot of a percentage of the selected molecules in FIG. 14(a) after 30 iterations of the random forest model that are in the top x of molecules in the test set according to minimizing the relative free binding energy.

### DETAILED DESCRIPTION

Molecular or drug design can be considered a multi-dimensional optimization problem that uses the hypothesis generation and experimentation cycle to advance knowledge. Each compound design can be considered a hypothesis which is falsified in experimentation. The experimental results are represented as structure-activity relationships, which construct a landscape of hypotheses as to which chemical structure is likely to contain the desired characteristics. The process of drug design is also an optimization problem as each project starts out with a product profile—e.g., target function—of desired, specified attributes. However, even though the objective can be accurately described, it has previously been an expensive and difficult challenge to find an optimal solution. One particular difficulty with this type of problem is to effectively construct the landscape of hypotheses across the vast space of feasible solutions from a relatively limited knowledge base of experimental results.

The drug discovery process is typically performed in iterations known as design cycles. At each iteration a set of molecules or compounds is synthesized, and their biological properties are measured. The activities are analyzed, and a new set of compounds is proposed, based on what has been learned from previous iterations. This process is repeated until a clinical candidate is found. As well as activity, the measured biological properties can include one or more of selectivity, toxicity, affinity, absorption, distribution, metabolism, and excretion.

At any particular stage of the process, a set of compounds will have been synthesized or made, with their biological activity being known. An aim of the process is to find one or more optimal compounds from a large population or pool of compounds that could be synthesized, but for which there are only resources and/or time to synthesize a subset of compounds from the population.

An automated or computational drug design process uses a mathematical model, such as a machine learning (ML) model, to predict or hypothesis which compounds in the population of compounds that could be made are optimal compounds, e.g., those compounds that maximize (or minimize) a particular/desirable biological activity.

Active Learning is a special case of machine learning in which a learning algorithm can interactively query a user—or some other information source—to label new data points with the desired outputs. One use case for this technique is when unlabeled data is abundant but manual labelling is expensive, which is a common scenario in drug discovery.

The ML model is trained using the available structure-activity relationships from experimental results, i.e., from those compounds in the population that have already been synthesized and tested. The strategy or approach of using an ML model to select for synthesis those compounds with the highest predicted activity (or other desirable target property) from the population of possible compounds is referred to as ‘exploitation’. An exploitation strategy may be regarded as a use phase of the process. Various mathematical approaches may be utilized to provide an ML model that performs exploitation. For instance, these include support vector machine algorithms, neural networks, and decision trees.

The exploitation approach will only be successful if the predictive capability of the ML model is sufficiently accurate (i.e., if the ML model is sufficiently well trained). Each compound from the population that is synthesized and tested is added to a training set of compounds that is used to train the ML model. The number of molecules or compounds that are added to the training set at a particular iteration is typically constrained by resource. That is, the number of compounds in the subset of compounds that is synthesized at each iteration will typically be defined at a prescribed maximum number.

The predictive capability of the ML model will be sufficiently accurate only if there is a sufficient number of compounds in the training set. As such, a certain number of iterations or design cycles may need to be performed—in which the prescribed maximum number of compounds are added to the training set at each iteration, for instance—before the ML model is sufficiently trained.

Also, the predictive capability of the ML model will be sufficiently accurate only if the compounds in the training set are sufficiently representative of the overall population of compounds that can be selected for synthesis. It is therefore important that, prior to the ML model being sufficiently well trained, compounds that will be most helpful in improving the ML model—i.e., those that will be most representative—are included in the subset to be synthesized at any given iteration. Selecting compounds for synthesis on this basis is referred to as ‘exploration’. Several approaches are known for selecting compounds for synthesis as part of an exploration strategy, for instance techniques based on distance metrics between compounds in a population, or based on diversity of compounds in a population in terms of chemical structure. An exploration strategy may be regarded as a learning phase or training phase of the process.

Exploitation and exploration strategies therefore have competing needs when selecting a subset of compounds for synthesis at a particular iteration of a drug discovery process. Indeed, a choice as to which strategy is appropriate will likely change in dependence on the particular stage of the drug discovery process. For instance, at an early stage of a drug discovery project, it is less likely that a sufficiently well-trained model has yet been built. An exploration strategy at this stage may therefore be the most appropriate strategy as the reward of exploration is ultimately a better-trained, and therefore more accurate, model. An exploitation strategy would not make best use of limited resources at this stage as exploitation is not a particularly good strategy for increasing the representativeness of the training set. On the other hand, if the ML model is already sufficiently well-trained—for instance, at a later stage of a drug discovery project—exploitation would be the appropriate strategy in that case as the subset of compounds selected by the model for synthesis is more likely to be optimal compounds relative to desired characteristics, e.g., high biological activity levels. At this stage, an exploration strategy would not make best use of the limited resources as exploration is not an optimal strategy for selecting compounds that are likely to have desired characteristics.

As mentioned above, a ML model for performing an exploitation strategy will only (be likely to) make accurate predictions if: there are a sufficient number of compounds in the set used to train the ML model; and the compounds in this training set are sufficiently representative of the pool of compounds from which compounds to synthesize are to be selected. The first of these means that a certain number of design cycles may need to be performed to obtain a sufficient number of synthesized compounds (unless data relating to a sufficient number of previously-synthesized compounds is already available). The second of these means that, for initial design cycles in the early stages of a drug discovery project, it may not be desirable to base a decision on which compounds to include in a set to be synthesized (solely) using a ML model that can only perform exploitation. This is because such a ML model will predict which compounds are highly active according to a model which has not yet been trained to a sufficient level, meaning that the predictions will be less likely to be accurate. In addition, synthesizing compounds according to such a prediction will not be of use in improving the ML model for the subsequent design cycle, as the ML model prediction further focusses in on the relationships/information already identified from the training set of compounds. In particular, a prediction from a ML model that purely performs exploitation does not assist in suggesting which compounds to synthesize with the aim of improving the accuracy of the ML model for the next design cycle.

In order to reduce the time and cost associated with a drug discovery project, the number of iterations or design cycles that is needed to discover a candidate or optimal compound having the desired properties should be minimized. It is therefore critical that a sufficiently well-trained model for predicting compounds having the desired properties can be built as quickly as possible, i.e., requiring as few compounds in the training set as possible. As such, it is important that the most representative compounds are selected for synthesis in the early stage of a project to minimize the number of iterations where (at least a degree) of exploration is needed, as a candidate compound is unlikely to emerge from iterations employing such a strategy.

The present invention is advantageous in that it provides an improved computational drug design method for designing and using a machine learning model for identifying a candidate compound from a population of compounds as part of a drug discovery process. In particular, the invention advantageously provides a machine learning model that can incorporate and perform both exploitation and exploration strategies, separately or in parallel. The invention advantageously allows for the optimization and selection of multiple compounds in parallel for synthesis at a given design cycle of a drug discovery project, and the invention advantageously allows for the optimization of compounds against multiple design objectives defining various desired biological properties of a candidate compound. The invention also provides a more flexible method for incorporating various preferences (of a chemist, for instance) in respect of objectives to be achieved or optimized by a candidate compound of a particular drug discovery project, and/or in respect of differentiating between compounds that each satisfy the various objectives when choosing which compounds to synthesize.

In accordance with the present invention, a step of a computational drug design method is to define a population of a plurality of compounds or molecules. In particular, this population is the set of compounds that can be selected for synthesis during a particular drug discovery project. The population can be defined or acquired in any suitable manner, e.g., via known computational methods and/or with human input. For instance, the population may be a set of compounds obtained from a generative or evolutionary design algorithm. In particular, an evolutionary design algorithm may generate a number of novel compounds based on an initial set of one or more known compounds—e.g., an existing drug—that have at least some of the desired properties of an optimal compound for a particular project on which the present method is to be used. Alternatively, a number of novel compounds may be generated in any suitable manner. Those generated novel compounds having at least some desired features may be retained for further analysis. In one example, a starting group of compounds (including millions of compounds, for instance) may be reduced in number by applying known methods to keep certain ones of the compounds with at least some desired features for a particular project at hand. One or more filters may be applied to the retained compounds to remove any undesirable compounds. The filters can be defined according to any appropriate criteria for selecting (or filtering) desirable compounds from undesirable compounds. For instance, one useful filter may be adapted to remove duplicate compounds. Another filter may be adapted to remove compounds having a certain level of toxicity. The filtered set of compounds may then form the population from which selection for synthesis may be made.

The population may include any suitable number of compounds. Generally, the population will include more—and likely significantly more—compounds than a number of compounds that can be synthesized as part of the particular drug discovery project, e.g., for reasons of available resource. However, the population will also generally not include so many compounds such that computational analysis of the population according to the present invention is not feasible. For instance, the number of compounds in the population may typically be of the order of hundreds or thousands of compounds, but it will be understood that for any given project the population may be larger or smaller than this.

Each compound in the population includes a number of structural features that combine to form its chemical structure. Such structural features can be represented in any suitable manner. For instance, one way in which to describe the structure of a compound or molecule is via fingerprinting. In particular, the fingerprint of a particular compound may be represented as mathematical objects—e.g., a series of bits or list of integer numbers—that reflect which particular structural features or substructures (fragments) are present or absent in the compound.

There are several different classes of fingerprints, such as topological fingerprints, structural fingerprints, and circular fingerprints. A common circular fingerprinting method is Extended Connectivity Fingerprinting (ECFP). A number of ECFP methods are known, such as ECFP0, ECFP2, ECFP4, ECFP6, ECFP8, ECFP10 and ECFP12. As is known in the art, determining a fingerprint of a compound will generally include assigning each atom in a compound with an identifier, updating these identifiers based on adjacent atoms, removing duplicates, and then forming a vector from the list of identifiers.

A next step of the computational drug design method is to define a training set of compounds from the population. The training set includes those compounds in the population whose biological properties are known. That is, the training set includes those compounds from the population that have been synthesized and tested experimentally to determine certain biological properties, e.g., a biological activity. As such, the number of compounds in the training set increases as a drug discovery project progresses, i.e., as more iterations or design cycles are performed. At the start of the drug design project, relatively few compounds may be in the training set. For instance, the training set may include compounds for which biological properties are known a priori, e.g., compounds that have been previously tested as part of a different project, and which have at least some of the desired properties of an optimal compound according to the particular project under consideration.

Note that in order to perform the computational design method of the present invention, the training set needs to include at least some compounds. Therefore, if at the start of a drug design project none of the compounds in the defined population have been synthesized and tested, i.e., no biological properties of the population are known, the training set may be populated in any suitable manner as an initial step prior to training and executing a ML method (as described below) in accordance with the invention. For instance, compounds synthesized to provide an initial training set may be selected according to a different technique, e.g., a known exploration strategy, or simply at random from the population.

A next step of the computational drug design method is to define a plurality of objectives each defining a desired biological property. That is, the multiple objectives outline the desired biological properties that would be exhibited by a candidate compound for a particular drug design project. The objectives may be based on various biological properties exhibited by compounds, for instance on one or more of biological activity, selectivity, toxicity, absorption, distribution, metabolism, and excretion. Each objective may be defined relative to a particular biological property in any suitable manner. For instance, an objective may be simply to maximize or minimize a particular biological property. Alternatively, an objective may be to achieve a particular desired value for a particular biological property, or the objective may allow for a desired range of values for the particular biological property to be acceptable in a candidate compound, or may constrain the value of a particular biological property to be greater than, or less than, a certain threshold value. One or more objectives may be defined for any given biological property. Purely for illustrative purposes, an example of a profile of an ideal molecule or compound for a certain drug discovery project may be expressed in terms of the following objectives: activity against a primary target X as high as possible; lipophilicity (log P) between 2 and 6; and activity against an unwanted target Y (pIC50) strictly below 5.

The (ultimate) aim of an ML model used as part of the described computational design method is to suggest or predict one or more compounds from the population that satisfy the defined objectives. A next step of the computational drug design method is to use the defined training set of compounds to train such an ML model. In particular, the ML model is a Bayesian statistical model whose output is a probability distribution approximating biological properties of compounds in the population as an objective function of structural features of the compounds in the population.

Bayesian optimization is a useful method for optimizing a function whose form is unknown (i.e., a ‘black box function’), and for which evaluating the function at points of the input space is costly. Bayesian optimization may therefore be considered a useful approach in computational drug discovery. This is because the types of functional relationships between compounds in a population of compounds are not known a priori, and also because synthesizing and testing a compound, i.e., the evaluation cost, can be both time consuming and expensive.

Bayesian optimization is a class of ML-based optimization methods focused on maximizing/minimizing an objective function across a feasible set or search space. A number of further general assumptions for problems using Bayesian optimization are typically made, or are common to problems addressed using Bayesian optimization. For instance, the dimensionality of the input space is generally not too large, the objective function is generally a continuous function, a global maximum/minimum is sought, and no gradient information is given with evaluations of the function, thereby preventing optimization methods based on derivatives, such as gradient descent or Newton's method. In the context of drug discovery, it is clear that not all of these general assumptions are applicable. For instance, Bayesian optimization for drug discovery would be modelled on discrete space—with each discrete point representing a compound from the population—instead of continuous space. Also, a problem in the context of drug discovery may have an input space that is of relatively high dimension. In particular, each dimension of the input space may represent a particular structural feature or fragment that is present or absent from a given compound, and the representation of the compounds in a model may include thousands of different such structural features that are encoded as being present or absent in each case. It is clear, therefore, that some standard Bayesian optimization techniques may not be suitable for a computational method in the context of drug discovery as in the present case, and that suitable modifications may need to be made. This will be described in greater detail below.

Bayesian optimization uses a Bayesian statistical model, or surrogate, for modelling the objective function. In this case, the objective function describes relationships between biological properties of compounds in the population and the structural features of those compounds. The Bayesian statistical model provides a Bayesian posterior probability distribution that describes potential values of the objection function at a given point, e.g., a point that is a candidate for evaluation. Each time the objective function is evaluated/observed at one or more new points, the posterior probability distribution is updated. That is, each time a compound from the population is synthesized to determine its biological properties, this compound can then be used to update the model approximating the relationships between biological properties and structural features.

When applying Bayesian optimization to a problem, the model that is used generates a measure of uncertainty, i.e., a way to quantify how certain the model is of its own prediction. The Bayesian statistical model may be a Gaussian Process model, which includes such a measure of uncertainty. A Gaussian Process is a stochastic process—i.e., a collection of random variables indexed by time or space—such that every finite collection of those random variables has a multivariate distribution. That is, every finite linear combination of the random variables is normally distributed. Broadly, a Gaussian Process model assumes that all data, training or not, is generated from the same Gaussian Process, and this is typically a good approximation.

Gaussian Process regression is one type of Bayesian statistical approach for modelling functions. Whenever there is an unknown quantity in Bayesian statistics—for instance, a vector of the objective function's values at a finite collection of input points—it is supposed that it was drawn at random from nature for some prior probability distribution (or simply, ‘prior’). Gaussian Process regression takes this prior distribution to be multivariate normal, with a particular mean vector and covariance matrix.

The mean vector may be constructed by evaluating a mean function at each of the input points. One option is to set the mean function to be a constant value; however, other suitable forms for the mean function are possible, e.g., a polynomial function, when the objective function is believed to have some application-specific structure. The covariance matrix may be constructed by evaluating a covariance function or kernel at each pair of points. That is, when predicting the value for an unseen point—i.e., a point that has not been evaluated and so whose function values are not known—the model uses a measure of similarity between points, where this measure of similarity is provided by a kernel function. The kernel may be chosen so that points that are closer together in the input space have a larger positive correlation. This encodes a belief that their function values should be more similar than a pair of points that are further from one another in the input space. Therefore, training points—i.e., points that have been evaluated and whose function values are known—that are in the neighborhood of an unseen point weigh heavier on the prediction for the unseen point relative to training points not in the neighborhood.

Suppose, for instance, that a number of points in the input space have been observed, and that it is desired to predict the value of the objective function at a new point. The prior distribution may be determined using Gaussian Process regression and then a conditional distribution of the objective function at the new point may be calculated given the observed point using Bayes' rule (as is known in the art). This conditional distribution is referred to as the posterior probability distribution in Bayesian statistics. The posterior mean may be a weighted average between the prior and an estimate based on the known data (i.e., evaluated or observed points) with a weight that depends on the kernel. The posterior variance (i.e., uncertainty) may be equal to the prior covariance less a term that corresponds to the variance removed by observing the function at the points mentioned above.

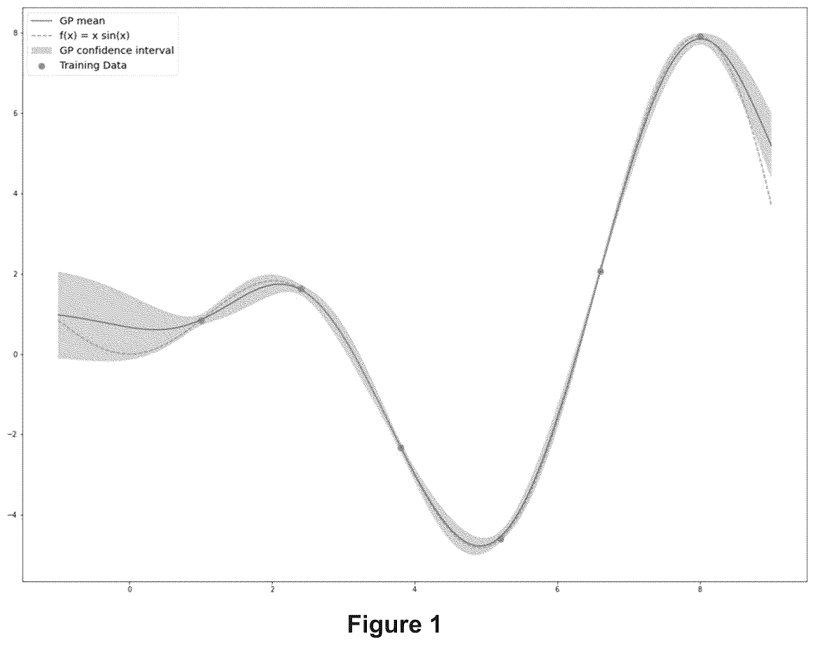

A simple example implementing the above approach is now provided for illustrative purposes. Consider the function f(x)=x sin x, and assume that six training points are provided to a Gaussian Process model that uses a radial basis function kernel. Then, predictions of the model are generated on the interval [0,10]. FIG. 1 shows a plot of the observed (training) points, the function f(x), the mean of the predictions, and the 95% confidence interval (twice the standard deviation, i.e., a measure of uncertainty). It may be seen that the uncertainty associated with predictions further away from the observed points is greater than that for predictions closer to the observed points.

As mentioned above, kernels typically have the property that the closer together points in the input space are to one another, the more strongly they are correlated, i.e., the more similar they are. However, a kernel needs to define how to measure how ‘close together’ a pair of points are in the input space. Typically, kernels are functions that depend on Euclidean distance. However, such kernels are less capable of dealing well with input points having high dimensionality. For instance, kernels based on measures of Euclidean distance may work sufficiently well where the input space is up to the order of tens of dimensions, e.g., 20 dimensions. However, as mentioned above, for analysis as part of an ML model a molecule or compound may be encoded/represented in a bit vector having a length of the order of thousands of bits, e.g., a 2048-bit fingerprint, where each bit is indicative of whether a particular structural feature or fragment is present or absent in a compound. That is, the input space in this context may be regarded as having thousands of dimensions. For instance, with a 2048-bit fingerprint, each fingerprint may be regarded as a vertex in a 2048-dimensional unit cube. Although a kernel based on Euclidean distance may be used in this context, it may not accurately reflect the difference between points in the input space—i.e., compounds in the defined population—as many of them will be equally far away from all of the others according to a measure of Euclidean distance.

In the context of the present invention, it may be beneficial to instead use a Tanimoto similarity as the basis for the kernel of the Gaussian Process model. The Tanimoto similarity or coefficient is a measure of the similarity and diversity of sample sets, and may be defined as the size of the intersection between sets divided by the size of the union of the sample sets. The Tanimoto coefficient is used in cheminformatics to determine the similarity between fingerprints. Beneficially, application of the Tanimoto coefficient in a kernel for a Gaussian Process model would not suffer from the above-described issues that would be experienced by Euclidean distance-based kernels for high-dimensional applications such as the present drug discovery use case. This is because the Tanimoto similarity may be regarded as being a cosine similarity, and so it may be regarded as a measure of angles rather than of distances (as is the case for Euclidean-based kernels).

The Bayesian optimization model also includes parameters of the prior distribution, referred to as hyperparameters. In particular, the mean function and kernel of the prior distribution includes hyperparameters. The choice/optimization of these hyperparameters is crucial because their influence can often be significant for various standard sample sizes. In the context of drug discovery, standard approaches to choose the hyperparameters of a Bayesian statistical model may not be suitable or optimal. One reason for this is because there is generally a relatively low amount of training data in the field of drug discovery. That is, the training set generally includes relatively few compounds with which to train the model. Of course, it is not necessarily feasible to add many, or any, further compounds to the training set as this requires relatively expensive and time-consuming synthesis and testing of compounds that have not yet been sampled. Another reason why some standard approaches to choosing the model hyperparameters may not be suitable in the context of drug discovery is because of so-called ‘activity cliffs’. That is, it may be relatively common to find that a pair of molecules having very similar, or almost identical, chemical structures exhibit a relatively large difference in terms of their respective activities. This significant difference in activity can be a result of a relatively small number of key atoms being added to, or removed from, a chemical structure. Such a phenomenon clearly needs careful attention in a model that predicts structure-activity relationships between compounds.

One way in which hyperparameters of a Bayesian statistical model may be chosen is by using a (type II) maximum likelihood estimation (MLE) approach. In particular, given a set of observations of the objective function—i.e., the training set of compounds with known biological properties in the present case—the likelihood of these observations under, or according to, the prior (which is dependent on the hyperparameters) is calculated. The likelihood is a multivariate normal density, and the hyperparameters are then set to the value that maximizes the likelihood in this distribution. A gradient descent method may be used to obtain the hyperparameters that maximize the likelihood of the observations under the prior. Both of these are issues when trying to use a model on unknown areas of chemical space where training data is sparse or absent.

In the context of drug discovery, using type II MLE to choose the hyperparameters may result in the model being steered towards low length scales because of the low amount of training data, meaning that a known point can influence the predictions for new points to a greater degree than is desired or optimal. Such an approach can also lead to high levels of noise in the model, and can result in the model overfitting the training data. Therefore, in order to scale and automate the training of a Bayesian statistical model for drug discovery without needing to manually check for these described issues, a more robust hyperparameter optimization approach is needed.

Another way in which hyperparameters may be chosen is using a cross validation approach. The general approach here is to split or partition the training set into a number of subsets; train the model using all but one of the partitioned subsets; and then test the model using the remaining (test) subset. This is then repeated for each of the different subsets as the test subset. This may be regarded as being a more robust way to train a ML model as it is the generalization capabilities of the model that are being optimized. However, a cross validation approach tends to be relatively computationally expensive, and slower to compute than type II MLE, for instance. The relatively large number of hyperparameters that need to be optimized in the context of drug discovery (because of the high dimensionality of the input data) means that a purely cross validation approach in this case would be prohibitively expensive in terms of computational expense.

In embodiments of the present invention, training the Bayesian statistical model may include tuning or training the hyperparameters of the model by applying a combination of a maximum likelihood estimation technique and a cross validation technique. By combining these two approaches or techniques, improved training of the hyperparameters may be achieved but at relatively small computational expense.

In one way, this combination approach may be regarded as being somewhat analogous to an ‘early stopping’ technique. ‘Early stopping’ is a machine learning technique, where a model is trained in steps via gradient descent. Every step, or every few steps, the model's performance is evaluated, usually on a set of data that has been held out called a validation set. If the performance has decreased since the previous time it was evaluated, then the model stops training in order to avoid overfitting of the training data. However, most models cannot be truly evaluated on the validation data unless it has never seen it. This means that, in practice, the model needs to be trained using less data than is actually available (in order to stop the model from overfitting).

For a Bayesian statistical (Gaussian Process) model in the context of drug discovery (i.e., operating on molecular data), the following approach may be useful. For the initial hyperparameters and priors on those hyperparameters of the model, it may be useful to start with a relatively high prior on the noise in the data. This is to ensure that activity cliffs (mentioned above) in the molecular data do not produce numerical errors or poor fitting. Then a standard gradient descent step of a maximum likelihood estimation approach may be performed through the model (e.g., with a Tanimoto kernel) on the entire training set, i.e., all of the compounds for which biological properties are known. A cross validation step may then be performed every few steps of the gradient descent, where the number of steps performed between cross validation can be selected as required. This is possible because of the particular property of Gaussian Process models that the covariance matrix that is used to compute the predictions depends only on its hyperparameters and the initial training data. Hence, the covariance matrix with a few rows and columns deleted is the same as the covariance matrix that would be obtained by first deleting the corresponding few data points from the training set. This means that, for a model with a covariance matrix that has already been determined, a set number (e.g., 10, or any other suitable number) of rows and columns can be hidden away, but a model that has the same hyperparameters and all but the number of training points that corresponding to the hidden rows and columns. Then this smaller model may be validated by predicting on the hidden points to obtain a particular metric of interest (e.g., ‘R squared’ for regression). If, instead, this process is performed on k-folds (where k is the number of subsets that the training data is split into)—that is, hiding the first 1/k of the data and predicting on it, then on the second 1/k of data, etc.—then a more accurate estimate of the generalization power of the model is obtained while, crucially, using the entire training set for gradient descent. As small training sets are the norm for drug design, then it cannot be afforded to use some (e.g., 10 out of 50, or any other suitable number) of compounds in the training set to ensure that the model does not overfit. Tuning a Gaussian Process model in the above manner avoids this issue. Another advantage is that model validation is given at almost no computational cost.

In Bayesian optimization, once the Bayesian statistical model—e.g., Gaussian Process model—has been trained to model the objective function using the training set, an acquisition function is used to determine at which points of the input space the function should be evaluated, sampled or observed next. In particular, an acquisition function is a useful tool in Bayesian optimization that shifts the problem from finding a global maximum in an intractable objective function, to finding the global maximum of a continuous, differentiable, fast-to-compute function. An acquisition function may be regarded as a map from a distribution and a state to a real value. The distribution may be a normal distribution, and the state may include values such as the maximum function value obtained thus far, the remaining budget of points for evaluation, etc.

An acquisition function uses the output from the Bayesian statistical model—in particular, the predicted mean and variance of the posterior probability distribution—to direct the search across the input space. The use of an acquisition function with a Bayesian statistical model allows for a trade-off between an exploitation approach and an exploration approach to be included in the predictions provided by the ML model. This is because the predictions include both mean values and variance values. By focusing on areas of the input space with high mean values, but penalizing higher variance values, exploitation of the current model is achieved. On the other hand, by focusing on areas of the input space with high variance values, the search is biased towards unexplored regions of the input space with few, if any, observed points, and as such exploration of the input space is achieved. Acquisition functions have tuning parameters that can be set according to a desired balance or trade-off between exploitation and exploration of the model at a particular design or iteration.

One type of acquisition function is an expected improvement function. This type of acquisition function selects as the next point for evaluation the point in the input space which has the highest predicted or expected improvement over the current highest value of the function in the training set of observed points. Another type of acquisition function is a probability of improvement function. This selects as the next point for evaluation the point in the input space which has the highest probability of showing an improvement over the current highest value of the function in the training set. A further type of acquisition function is a lower or higher confidence bound function, which selects the next point with reference to the current variance or standard deviation of the posterior mean. For instance, a lower confidence bound acquisition function may consider a curve that is two standard deviations below the posterior mean at each point, and then this lower confidence envelope of the objective function model is minimized to determine the next sample point. As mentioned above, expressions for each of these acquisition functions include weighting or tuning parameters that can be tuned according to a desired balance between exploitation and exploration approaches when selecting the next point to be observed. The acquisition function may depend on the posterior mean and variance values of the posterior distribution. A weighting parameter on the posterior mean term of the acquisition function may be used to set a desired level of exploitation, and a weighting parameter on the posterior variance term of the acquisition function (relative to the mean weighting parameter) may be used to set a desired level of exploration. Such weighting parameters may be user-defined to set the desired strategy.

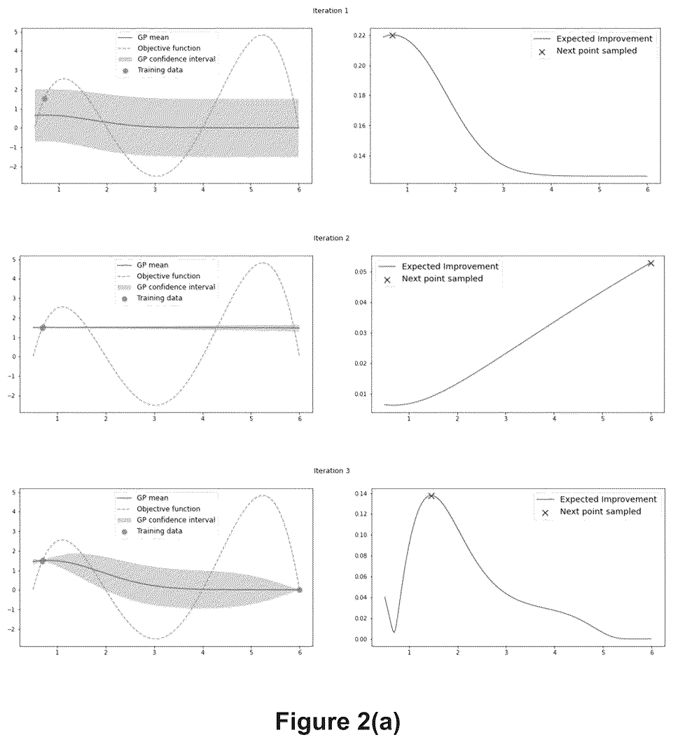

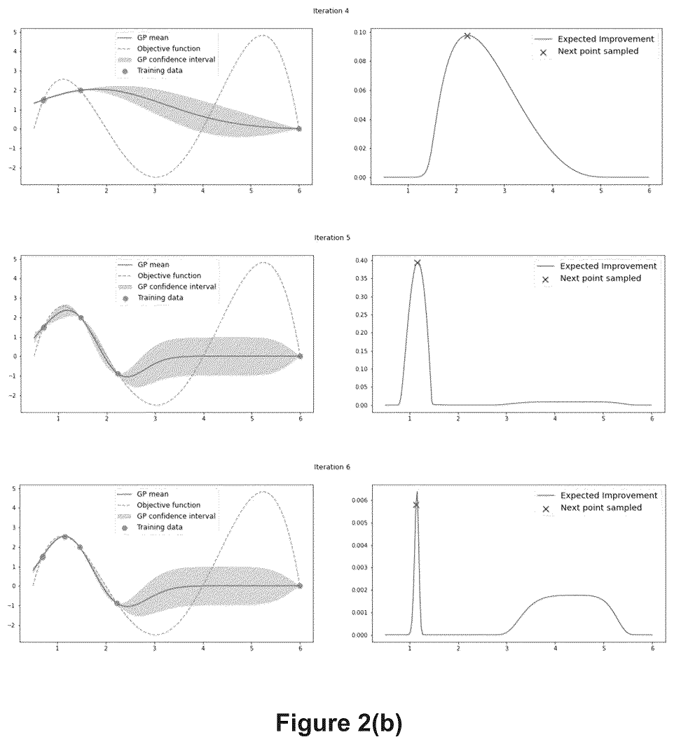

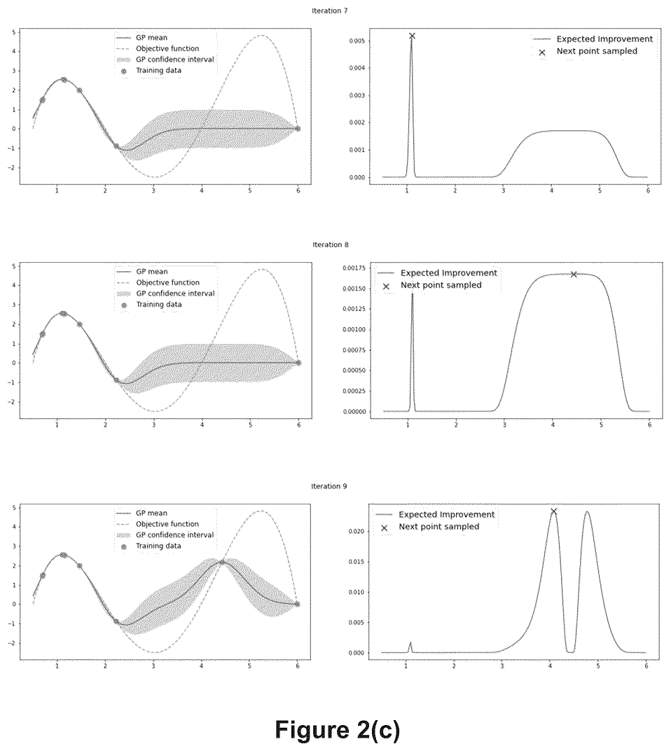

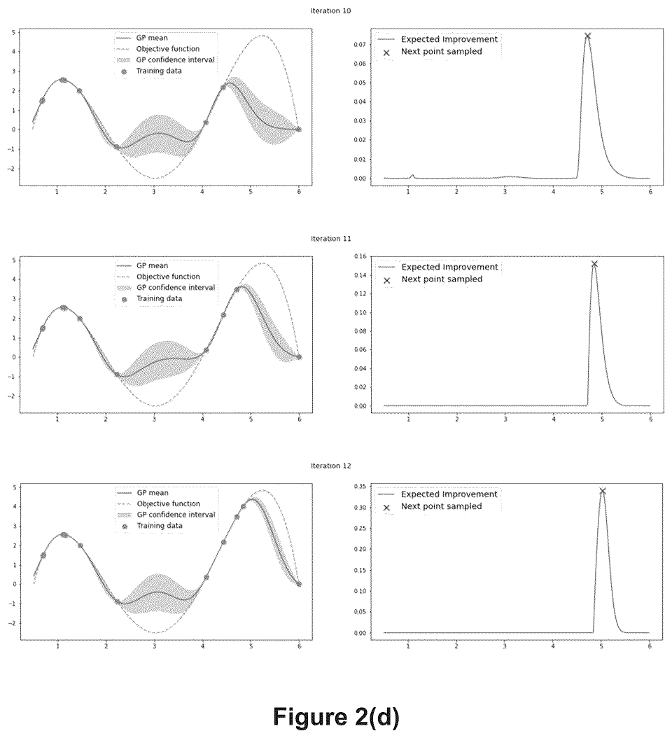

FIG. 2 illustrates an example of how a surrogate function, e.g., Gaussian Process model, is modelled using sampled points in order to optimize an objective function. At each iteration of the process, an acquisition function is optimized in order to select the next point to sample or evaluate. As more sampled points are available at each subsequent iteration, the surrogate function becomes more accurate, and the selected next sampling point becomes more likely to maximize the objective function.

Bayesian optimization techniques typically may be used to select a single point at which to evaluate the unknown objective function next. However, as mentioned above, in a drug discovery project it is typically the case that multiple compounds are selected for synthesizing and testing at any given design cycle for reasons of efficiency. That is, multiple points need to be selected for evaluation at a given iteration. Therefore, according to a step of the computational drug design method, a subset of a plurality of compounds from the population which are not in the training set is determined or selected. In particular, the subset is determined according to an optimization of an acquisition function based on the probability distribution from the trained Bayesian statistical model and based on the defined plurality of objectives. That is, the method automatically chooses a plurality of compounds to be sampled at a given iteration or design cycle. The number of compounds that the method chooses for inclusion in the subset may be user-defined, for instance according to available levels of resource to synthesize and test a certain number of compounds at a given design cycle. The size of the subset may be the same for each iteration (i.e., each time the computational drug design method is iterated), or may be changed for different iterations, depending on requirements.

In order to determine the subset, the Bayesian statistical model may be trained, and the acquisition function may be optimized, successively to choose one compound at a time until the required number of compounds for the subset have been selected. In particular, after the Bayesian statistical model has been trained on the training set, one compound from the population that is not in the training set may be identified by optimizing the acquisition function based on the probability distribution from the trained Bayesian statistical model and based on the defined plurality of objectives. This first selected compound needs to be taken into account when repeating the optimization to find a second compound for the subset. However, as the biological properties of the first selected compound are unknown, then dummy or fake labels may be applied to the first selected compound as a proxy of its biological properties. By virtue of the dummy labels, the predicted variance around the identified compound will be lowered. The method may then involve retraining the Bayesian statistical model using the dummy labels of the first selected compound (as well as the training set of compounds), and then a second compound from the population that is not in the training set may be identified for the subset by optimizing the acquisition function based on the probability distribution from the retrained Bayesian statistical model and based on the defined plurality of objectives. The second selected compound may then similarly be given dummy labels so that the Bayesian statistical model may be further retrained. In particular, the method may include repeating the steps of: retraining the Bayesian statistical model using the training set of compounds and the one or more identified compounds thus far; and optimizing the acquisition function based on the probability distribution from the retrained Bayesian statistical model and based on the defined plurality of objectives to identify another compound for the subset. Specifically, these steps may be repeated until the desired number of compounds have been identified for the subset.

The fake or dummy labels or biological property values for each identified compound for the subset may be set or determined in any suitable manner. For instance, the dummy labels may be set according to a kriging believer approach, which sets dummy values based on the predicted values of the biological properties from the Bayesian statistical model, optionally varied to incorporate upper and lower bounds to reflect a degree of optimism or pessimism regarding the prediction. Alternatively, the dummy labels may be set according to a constant liar approach, where the relevant values or labels may be set to be constants, regardless of the point. For instance, the mean of the model may be such a suitable constant.

A different approach (from the sequential selection with dummy labels approach above) could be used. For instance, a batch of compounds may be selected using a multipoint expected improvement (q-EI) approach. In such an approach, the expected increase from the current best solution is computed, conditioned on a set of points (rather than a single point). An appropriate approximation for discrete space then allows for such a multi-point acquisition function of multi-point decision strategy may then be implemented.

Many Bayesian optimization techniques may typically be used to optimize a single parameter of the function, i.e., a single objective. However, as described above, there will typically be a number of criteria against which a compound needs to be optimized in order to be a suitable candidate compound. That is, multiple parameters of the function need to be optimized according to various desired properties of a candidate compound of the particular drug discovery project under consideration, i.e., the optimization aims to achieve a plurality of objectives in parallel. The objectives will also often be conflicting. In addition, in the context of drug discovery, preference of objectives is not monotonic (unlike in some other applications).

The probability distribution from the Bayesian statistical model may therefore be a multi-dimensional distribution. In particular, the multi-dimensional distribution may include a (one-dimensional) distribution for each biological property associated with each respective one of the plurality of objectives. One option to optimize these multiple distributions in parallel relative to their respective objectives is to use a multi-dimensional acquisition function. Each dimension of the acquisition function may correspond to a respective objective. For instance, in such a case the multi-dimensional acquisition function may be a hypervolume expected improvement function.

Another option to optimize against multiple objectives in different dimensions is to transform the problem into a one-dimensional problem. In particular, one or more aggregation functions may be used to simplify the problem of multi-objective optimization. Such aggregation functions take the mean and variance for each dimension (i.e., each biological property with a corresponding objective) from the Bayesian statistical model as input. The output is then a one-dimensional distribution with a mean and variance. That is, the uncertainties in the predictions of the model are carried through the aggregation function to be leveraged by the acquisition function. Furthermore, input to the aggregation function can be readily extended to any required number of dimensions. Advantageously, the optimization can then be performed using a one-dimensional acquisition function, which is typically simpler to execute. For instance, such an acquisition function may be an expected improvement, probability of improvement, or confidence bounds function, as mentioned above. Statistical independence between each pair of dimensions may be assumed to apply the aggregation function. The aggregation function may include one or more of a sum, mean, geometric mean, and a product function or operator (each of which may be weighted to enable preference over individual components), for instance using one or more of the following results.

For any random variables X, Y:

[symbol]

[X+Y]= [symbol] [X]+ [symbol] [Y]

For any independent random variables X, Y:

[symbol]

[X+Y]= [symbol] [X]+ [symbol] [Y]

[symbol]

[XY]= [symbol] [X] [symbol] [Y]

[symbol]

[XY]= [symbol] [X²] [symbol] [Y²]−( [symbol] [X])²( [symbol] [Y])²

These results can generalize to N variables, as well as to scalar multiplication using basic expectation and variance properties.

In the case of general functions and correlated inputs, where the above results may not hold, a Monte Carlo sampling technique may be used, for instance. In particular, after determining a correlation between inputs empirically, and samples are obtained from a multivariate distribution, the aggregation function may be determined for these samples. The mean and standard deviation may then be deduced from the results. The one-dimensional result of the aggregation may then be provided to a one-dimension acquisition function.

As different ones of the multiple objectives of the optimization problem may conflict with each other—i.e., optimizing against one objective has a detrimental effect on another objective—the optimization of the acquisition function based on the defined plurality of objectives may provide a Pareto-optimal set of compounds. One or more of these compounds then need to be selected for inclusion in the determined subset. This may be performed in any suitable manner, e.g., according to user-defined preference or desirability.

One way in which to deal with conflicting objectives and break ties between compounds in the multi-objective optimization is to encode preferences into the optimization. This may be achieved via the application of utility functions to the posterior priority distributions associated with the respective objectives. In a case where a user has an order of preference over a set of choices, a utility function can be used to encode that preference by assigning real numbers to each of the alternatives. Hence, for each of one or more of the objectives, the method may include mapping a preference—which may be a user-defined preference—associated with the biological property, or the distribution, of the respective objective by applying a respective utility function to the probability distribution from the Bayesian statistical model to obtain a preference-modified probability distribution. Optimization of the acquisition function may then be based on the preference-modified probability distribution. It is crucial that the uncertainty associated with the prediction from the model is propagated through to application of the acquisition function, and the utility functions (as well as the aggregation functions described above) are advantageous in that the uncertainty is retained in their output.

In some cases, the defined preference may be indicative of a priority of the respective objective relative to other ones of the plurality of objectives, e.g., if it is more critical to meet one objective relative to another for the purposes of obtaining a candidate compound.

Preferences may also be introduced based on the particular predictions of the model. For instance, preferences may be encoded in favor of predictions over which the model has greater certainty. That is, for one of the biological properties of one of the compounds, it may be the case that the lower an uncertainty value associated with the probability distribution for the biological property, the greater the preference associated with the respective biological property. In this way, the uncertainty of the model prediction is useful not only as an output of the utility functions (to be used by the acquisition function), but also as an input. Purely as an illustrative example, suppose the plurality of objectives are defined to optimize against a number of activity objectives, as well as lipophilicity (log P) needing to be strictly between 0 and 2 (where any value between 0 and 2 is equally desirable). Consider a case in which the Bayesian statistical model prediction returns two compounds, X and Y, having the same predictions for activity, the same log P mean prediction, and a log P standard deviation of 0.5 and 3, respectively. In this case, compound X is the preferred compound as it is more likely to be in the desired range between 0 and 2 for the lipophilicity. In this case, if prediction uncertainty is not taken into account, the average utility function values would be identical, meaning that the method could not distinguish between X and Y even though a user would have a clear preference.

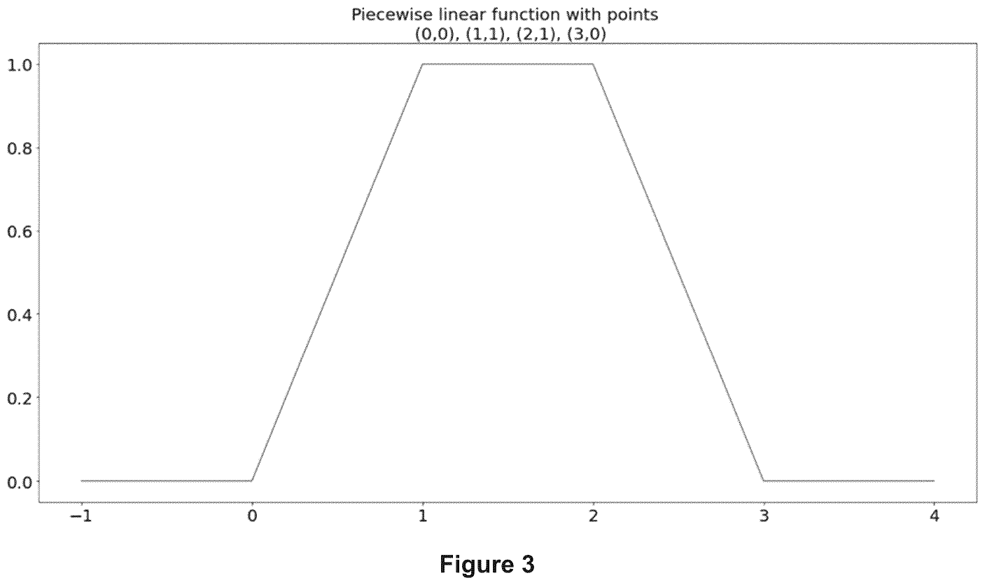

In practice, in the order of preference over a set of choices, those choices that are close together in the order tend to have similar levels of preference. Also, when the choices are real numbers, the utility functions may be continuous. The utility functions of the present method may advantageously be modelled as piecewise functions and, in particular, piecewise linear functions. That is, functions that, when plotted, are composed of straight-line segments defined as:

$$f ( x ) = \{ \begin{matrix}{a}_{0} x + {b}_{0} , & x \leq {x}_{0} \\ {a}_{1} x + {b}_{1} , & x \leq {x}_{1} \\ … & \\ {a}_{N - 1} x + {b}_{N - 1} , & x \leq {x}_{N - 1} \\ {a}_{N} x + {b}_{N} , & x \geq {x}_{N - 1}\end{matrix}$$

where [(a₀, b₀), (a₁, b₁), (a_{N}, b_{N})] are the N+1 linear functions and [x₀, x₁, . . . x_{N-1}] are the points between two consecutive lines. FIG. 3 shows an example of a piecewise linear function that may be used as part of the described method to include a degree of preference over predictions for different compounds.

Piecewise linear functions can be used in combination with normal distributions. In the present computational drug design method, the Bayesian statistical model provides predictions as normal distributions, which may then be passed to the piecewise linear utility functions. As mentioned above, the uncertainty in the normal distributions needs to be preserved through the utility functions (to be used subsequently by the acquisition function(s)). Given the prediction as a normal distribution, and the utility function outlined above, the mean and standard deviation may be determined. The following result is used to determine these values.

Let X ˜ [symbol] (μ, σ). The probability density function (pdf) for X is:

$$p ( X ) = \frac{1}{σ \sqrt{2 π}} {e}^{- \frac{1}{2} {( \frac{X - μ}{σ} )}^{2}}$$

For any random variable X with pdf pₓ and f a function:

[symbol]

[f(X)]=∫_{-∞}^{∞}f(x)pₓ(x)dx

The error function erf is defined as:

$$erf ( x ) = \frac{2}{\sqrt{π}} {\int }_{0}^{x} {e}^{- {t}^{2}} dt$$

For any normal distribution X with mean μ and standard deviation σ, its cumulative density function (cdf) is:

$${cdf}_{x} ( x ) = Φ ( \frac{x - μ}{σ} ) = {\int }_{- ∞}^{\frac{x - μ}{σ}} \frac{1}{\sqrt{2 π}} {e}^{- \frac{{t}^{2}}{2}} dt = {=}^{( 4 )} \frac{1}{2} [ 1 + erf ( \frac{x - μ}{σ \sqrt{2}} ) ]$$

The standard deviation of X can be written in terms of expected values:

σ(X)=\sqrt{ [symbol][X²]−( [symbol] [x])²}

### Expected Value

From the expression for [symbol] [f(X)] above:

$$[ f ( X ) ] = {\int }_{- ∞}^{∞} f ( x ) {p}_{X} ( x ) dx = {\int }_{- ∞}^{∞} f ( x ) \frac{1}{σ \sqrt{2 π}} {e}^{- \frac{1}{2} {( \frac{x - μ}{σ} )}^{2}} dx = {\sum }_{i = - 1}^{N - 1} {\int }_{{x}_{i}}^{{x}_{i + 1}} ( {a}_{i + 1} x + {b}_{i + 1} ) \frac{1}{σ \sqrt{2 π}} {e}^{- \frac{1}{2} {( \frac{x - μ}{σ} )}^{2}} dx$$

where x₋₁=−∞ and x_{N}=∞

For any a, b, μ and σ≠0:

$$\int ( a x + b ) \frac{1}{σ \sqrt{2 π}} {e}^{- \frac{1}{2} {( \frac{x - μ}{σ} )}^{2}} dx = \frac{\sqrt{\frac{π}{2}} σ ( a μ + b ) erf ( \frac{x - μ}{σ \sqrt{2}} ) - a {σ}^{2} {e}^{- \frac{1}{2} {( \frac{x - μ}{σ} )}^{2}}}{σ \sqrt{2 π}}$$

This result can be replaced above to obtain:

$$[ f ( X ) ] = {\sum }_{i = - 1}^{N - 1} \frac{\sqrt{\frac{π}{2}} σ ( {a}_{i + 1} μ + {b}_{i + 1} ) erf ( \frac{{x}_{i + 1} - μ}{σ \sqrt{2}} ) - {a}_{i + 1} {σ}^{2} {e}^{- \frac{1}{2} {( \frac{{x}_{i + 1} - μ}{σ} )}^{2}}}{σ \sqrt{2 π}} - {\sum }_{i = - 1}^{N - 1} \frac{\sqrt{\frac{π}{2}} σ ( {a}_{i + 1} μ + {b}_{i + 1} ) erf ( \frac{{x}_{i} - μ}{σ \sqrt{2}} ) - {a}_{i + 1} {σ}^{2} {e}^{- \frac{1}{2} {( \frac{{x}_{i} - μ}{σ} )}^{2}}}{σ \sqrt{2 π}}$$

### Standard Deviation

For any a, b, μ and a σ≠0:

$$\int {( a x + b )}^{2} \frac{1}{σ \sqrt{2 π}} {e}^{- \frac{1}{2} {( \frac{x - μ}{σ} )}^{2}} dx = \frac{1}{2} σ ( {a}^{2} ( {μ}^{2} + {σ}^{2} ) + 2 a b μ + {b}^{2} ) erf ( \frac{x - μ}{σ \sqrt{2}} ) -- \frac{a σ}{π \sqrt{2}} {e}^{- \frac{1}{2} {( \frac{x - μ}{σ} )}^{2}} ( a ( μ + x ) + 2 b )$$

From the above:

$${σ}^{2} ( f ( X ) ) = [ {f ( X )}^{2} ] - {( [ f ( X ) ] )}^{2} == {\int }_{- ∞}^{∞} {f}^{2} ( x ) \frac{1}{σ \sqrt{2 π}} {e}^{- \frac{1}{2} {( \frac{x - μ}{σ} )}^{2}} d x - {( [ f ( X ) ] )}^{2} == {\sum }_{i = - 1}^{N - 1} {\int }_{{x}_{i}}^{{x}_{i + 1}} {( {a}_{i + 1} x + {b}_{i + 1} )}^{2} \frac{1}{σ \sqrt{2 π}} {e}^{- \frac{1}{2} {( \frac{x - μ}{σ} )}^{2}} d x - {( [ f ( X ) ] )}^{2}$$

where x₋₁=−∞ and x_{N}=∞. By manipulation:

$${σ}^{2} ( f ( x ) ) = [ {f ( x )}^{2} ] - {( [ f ( x ) ] )}^{2} == {\sum }_{i = - 1}^{N - 1} ( \frac{1}{2} σ ( {a}_{i + 1}^{2} ( {μ}^{2} + {σ}^{2} ) + 2 {a}_{i + 1} {b}_{i + 1} μ + {b}_{i + 1}^{2} ) erf ( \frac{{x}_{i + 1} - μ}{σ \sqrt{2}} ) ) -- {\sum }_{i = - 1}^{N - 1} ( \frac{1}{2} σ ( {a}_{i + 1}^{2} ( {μ}^{2} + {σ}^{2} ) + 2 {a}_{i + 1} {b}_{i + 1} μ + {b}_{i + 1}^{2} ) erf ( \frac{{x}_{i} - μ}{σ \sqrt{2}} ) ) ++ {\sum }_{i = - 1}^{N - 1} ( \frac{{a}_{i + 1} σ}{π \sqrt{2}} {e}^{- \frac{1}{2} {( \frac{{x}_{i} - μ}{σ} )}^{2}} ( {a}_{i + 1} ( μ + {x}_{i} ) + 2 b ) ) -- {\sum }_{i = - 1}^{N - 1} ( \frac{{a}_{i + 1} σ}{π \sqrt{2}} {e}^{- \frac{1}{2} {( \frac{{x}_{i + 1} - μ}{σ} )}^{2}} ( {a}_{i + 1} ( μ + {x}_{i + 1} ) + 2 b ) ) - {( [ f ( X ) ] )}^{2}$$

Taking the square root of the above provides an expression for σ(f(X)).

Note that the last term ( [symbol] [f(X)])² is the square of the expected value expression computed above.

An analytical solution to computing the mean and the uncertainty through piecewise desirability functions has been found. Crucially, the equations can be vectorized, i.e., hold for X N-dimensional vectors of normal (N normally distributed variables instead of just one). This is important as vectorized operations—e.g., addition, multiplication, exponentiation, etc.—benefit from hardware acceleration, making them very fast to compute.

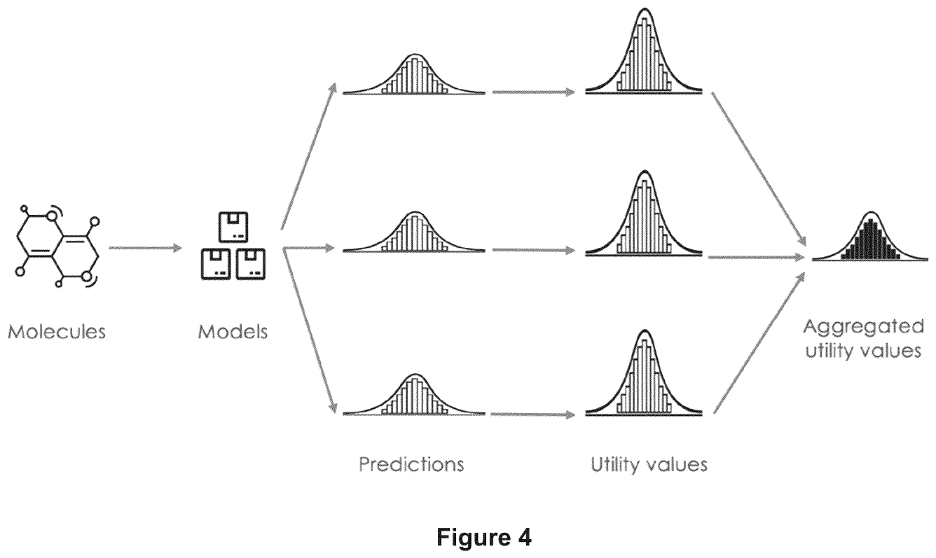

FIG. 4 schematically illustrates how compounds or molecules in the population may be fed to an ML model, i.e., Bayesian statistical model, that has been trained using those compounds from the population whose biological properties are known, i.e., the compounds in a training set. In the present multi-objective problem, the Bayesian statistical model may output multiple predictions (corresponding to the respective objectives) in the form of posterior probability distributions. Utility functions or values may then be applied to the respective predictions, e.g., to introduce preference into the predictions, while maintaining the uncertainty measures associated with the generated predictions. Aggregation functions or values may then be applied to the (preference-modified) predictions in order to reduce the dimensionality of the predictions to a single dimension, again while preserving the uncertainty associated with the predictions. The aggregated predictions may then be optimized using a one-dimensional acquisition function (optionally including a user-defined weighting according to a desired balance of exploitation versus exploration of the model) to select compounds for synthesis.

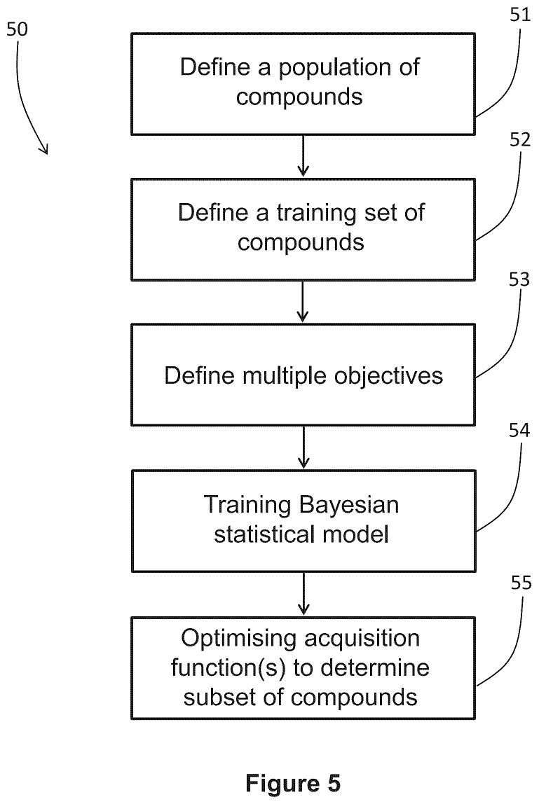

FIG. 5 summarizes the steps of a computational drug design method 50 in accordance with the invention. At step 51, a population of a plurality of compounds is defined, where each compound having one or more structural features. At step 52, a training set of compounds is defined. In particular, the training set includes those from the population for which a plurality of biological properties are known, e.g. those compounds that have previously been synthesized and tested. At step 53, a plurality of objectives is defined. In particular, each objective is indicative of, or defines, a biological property that would be exhibited by an ideal/candidate compound (for the specific drug discovery project under consideration). At step 54, a Bayesian statistical model, e.g., a Gaussian Process model, is trained using the training set of compounds. The Bayesian statistical model is then executed to output a posterior probability distribution approximating biological properties of compounds in the population as an objective function of structural features of the compounds in the population. The posterior probability distribution may be multiple posterior probability distributions, e.g., one corresponding to each of the multiple objectives. At step 55, a subset of a plurality of compounds is determined. In particular, the subset includes compounds from the population which are not in the training set. Specifically, the subset is determined according to an optimization of an acquisition function based on the probability distribution from the trained Bayesian statistical model and based on the defined plurality of objectives (i.e., to simultaneously optimize the plurality of objectives). That is, the compounds that best fit the optimization profile (e.g., ideal compound) are selected. The subset may be selected by repeating the model execution and acquisition function optimization steps a plurality of times to successively select one compound at a time for the subset, and retraining the model each time the steps are repeated (using fake labels for the compounds selected so far for the purpose of the training step). Optionally, one or more utility functions may be applied to the generated posterior probability distribution(s), prior to application of the acquisition function, to introduce user-preference regarding the objectives into the model predictions. Optionally, one or more aggregation functions may be applied to reduce the dimensionality of the generated model predictions prior to application of the acquisition function. At least some of the compounds in the determined subset may then be selected for synthesis and testing. These synthesized compounds may then be added to the training set for the next execution of the method 50, e.g., at a subsequent design cycle of the drug discovery project under consideration.

The method of the invention may be implemented on any suitable computing device, for instance by one or more functional units or modules implemented on one or more computer processors. Such functional units may be provided by suitable software running on any suitable computing substrate using conventional or customer processors and memory. The one or more functional units may use a common computing substrate (for example, they may run on the same server) or separate substrates, or one or both may themselves be distributed between multiple computing devices. A computer memory may store instructions for performing the method, and the processor(s) may execute the stored instructions to perform the method.

A comparison between the described Gaussian Process model and a standard random forest is now outlined and illustrated. A set of 14620 molecules with known biological activity PXC 50—in particular, hERG activities—is defined. Statistics of the data set are shown in Table 1.

TABLE 1

| | PXC50 |
|---|---|
| count | 14620.000000 |
| mean | 5.241873 |
| std | 0.887881 |
| min | −1.046000 |
| 25% | 4.568818 |
| 50% | 5.000000 |
| 75% | 5.722058 |
| max | 9.853872 |

The first 2000 molecules of the data set are used as the training data for training the models (in the manner described above for the Gaussian Process model). The performance of each model is then evaluated using the remaining molecules in the data set. The kernel used for the Gaussian Process model is a Jaccard kernel, which uses the Jaccard (or Tanimoto) distance between fingerprints.

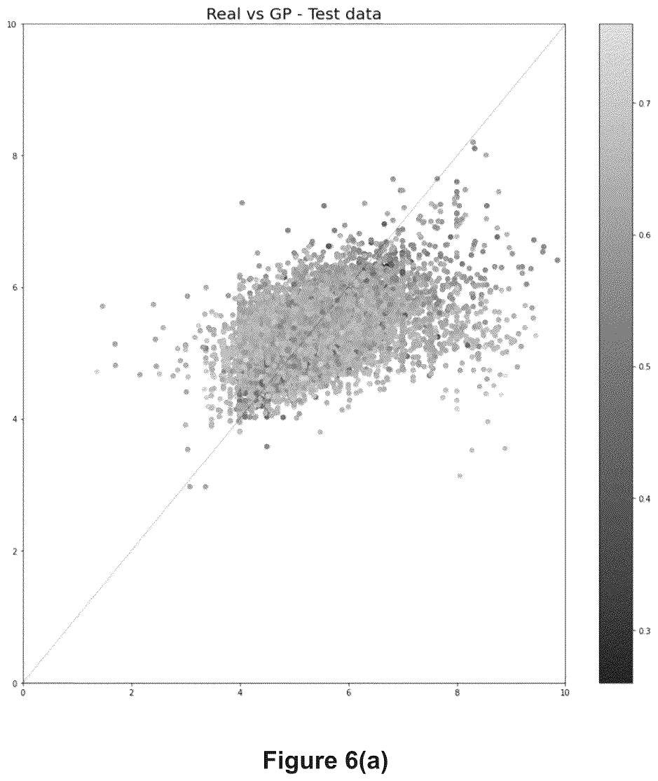

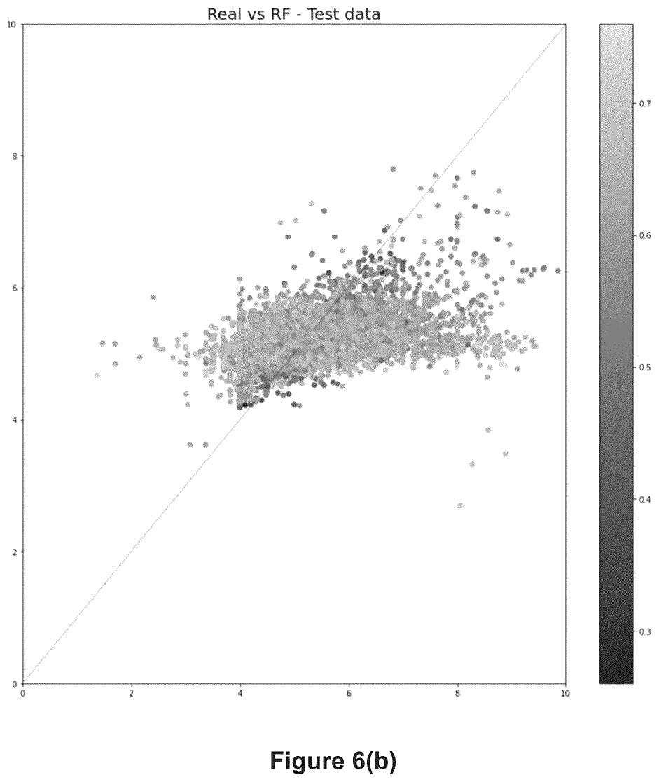

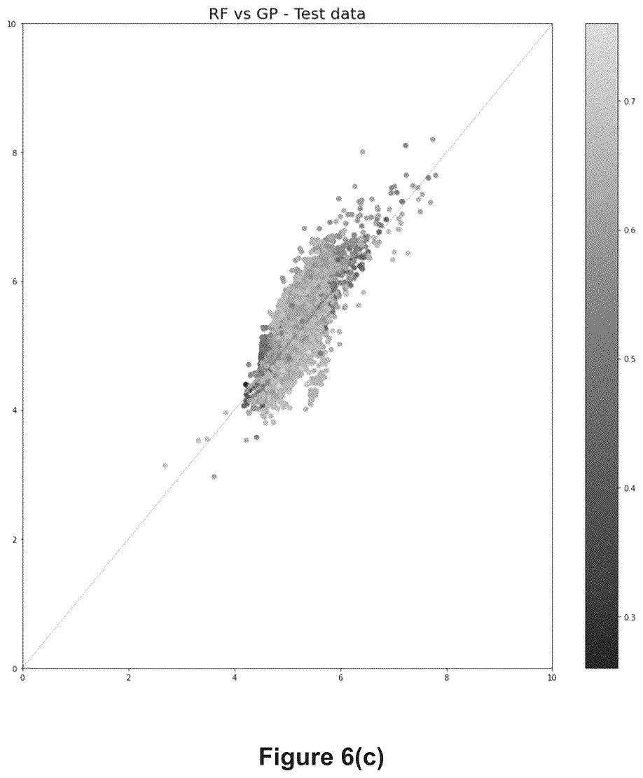

FIG. 6 compares the real, known biological activities of the molecules in the data set against the activities as predicted by the trained Gaussian Process and random forest models. In particular, FIG. 6(a) shows a scatter plot of the real activity values against those predicted by the Gaussian Process model for each of the molecules. Each dot—representing the molecules—has an associated degree of dependence on the variance of the Gaussian Process model. Similarly, FIG. 6(b) shows a plot of the real activity values against those predicted by the random forest model, and FIG. 6(c) shows a plot comparing the predicted activities obtained from the random forest and Gaussian Process models.

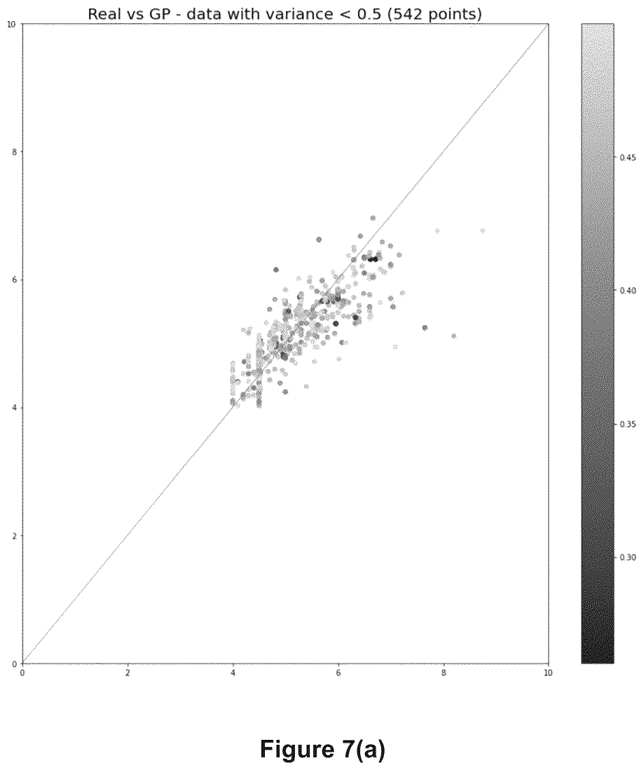

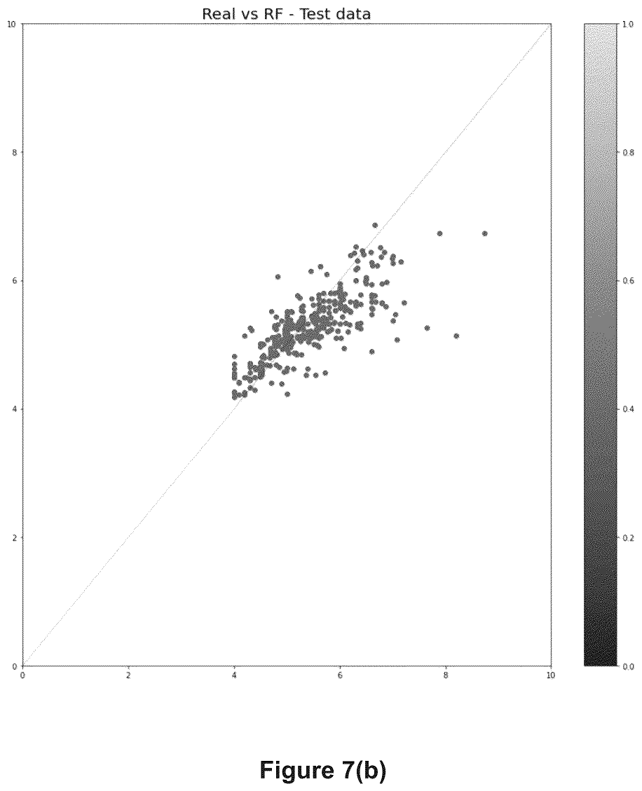

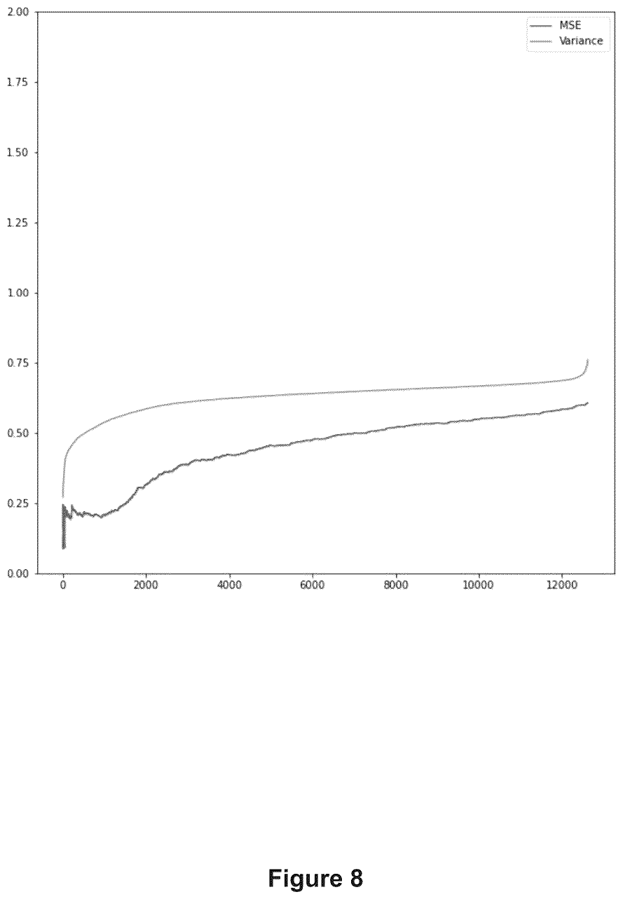

The variance threshold in the Gaussian Process model can be tweaked to illustrate how the certainty of the model correlates with accurate predictions. For instance, the model could be run with different upper thresholds for the variance, e.g., 1, 0.75, 0.6, 0.5, 0.4, or any other suitable value. FIG. 7(a) shows a scatter plot of the real activity values against those predicted by the Gaussian Process model with a variance threshold set to 0.5. For comparison, FIG. 7(b) shows a scatter plot of the real activity values against those predicted by the random forest model for those molecules as filtered for FIG. 7(a). Finally, FIG. 8 shows a plot of how the mean squared error (MSE) and the variance of the Gaussian Process model varies according to model certainty.

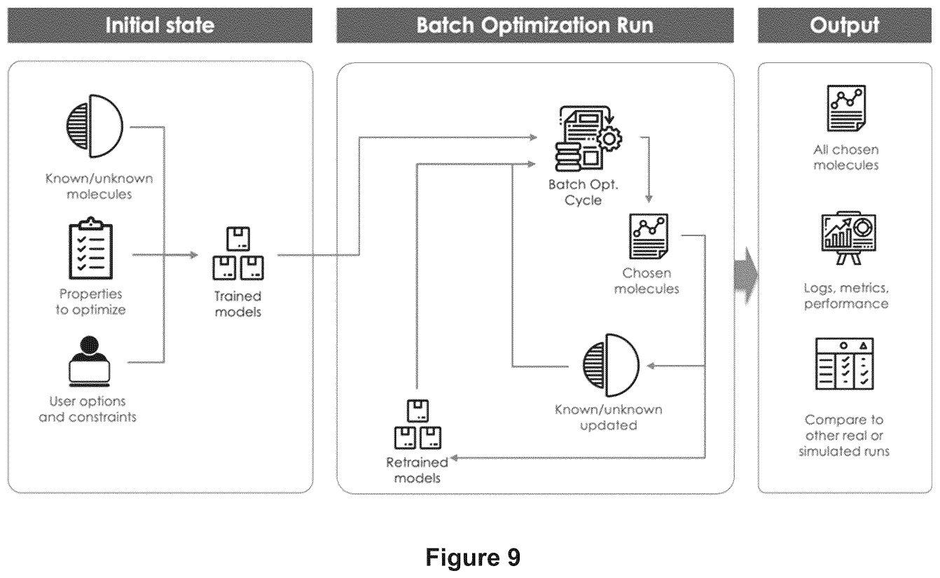

A further example is described in which multiple optimization cycles are simulated using the Bayesian optimization approach described above on existing, known sets of molecules to benchmark them. FIG. 9 schematically illustrates the main steps or modules for performing the benchmarking. In an initial state or stage, parameters to customize the simulation are set, e.g., by a user. Such parameters can include the acquisition function, the batch size, etc. The molecules that are already known to the model are set, as are the unknown molecules from which the model can choose. The plurality of properties or objectives are also set. In the batch optimization run stage, a single optimization step is performed (as described above) to select a batch of molecules. The model is then retrained by feeding the selected batch to the model with the correct labels, before a further optimization step may be performed. The output can include all of the selected molecules, and/or various logs/metrics associated with the model predictions.

One set of known molecules is the data set of 2500 compounds presented in Pickett et al., (2011) “Automated lead optimization of MMP-12 inhibitors using a genetic algorithm”, ACS Medicinal Chemistry Letters, 2(1), 28-33. This data set was generated by choosing a core with two R-groups. The core is fixed, and each R-group is essentially a placeholder, each for 50 different molecular structures to obtain 2500 combinations that contain that core. Of these combinations, only 1880 molecules were successfully synthesized and tested against an assay to yield a pIC50 value. Multiple cycles of synthesis may therefore be simulated by active/machine learning models (such as the one described above) or chemists with the aim of maximizing the pIC50 values found.

In one experiment, a number of chemists were given the same initial 14 compounds and the associated pIC50 values. With this information, the chemists were tasked with selecting another batch of 14 compounds, for which they are provided with the associated pIC50 values. This process continued for 10 batches (iterations), for a total of 140 selected compounds and 14 initial ones. Each chemist's performance was then evaluated based on whether a compound with the maximal pIC50 value had been found, the average pIC50 value on selected compounds, and the top N compounds selected. The described Gaussian process model was used to simulate the same experiment. In particular, the model was trained on the provided training data (i.e. the known pIC50 values). The Bayesian optimization algorithm selected a batch of compounds to optimize the objectives (i.e., maximize pIC50 value). The training set was then updated to include the selected compounds, the model was retrained, and the optimization was performed again. A comparison between the results of the present active learning approach and the best-performing chemist is presented in Table 2.

TABLE 2

| | No. of | | | | | |
|---|---|---|---|---|---|---|
| | invalid | Sum of | Average valid | Maximum | Minimum | Top 10 |
| | molecules | values | molecule value | value | value | values |
| Real | 24 | 690.9 | 5.9560 | 7.6 | 3.0 | [7.3 7.3 7.3 7.4 7.4 |
| candidates | | | | | | 7.4 7.5 7.5 7.6 7.6] |
| AL | 25 | 882.3 | 6.8395 | 8.0 | 3.0 | [7.5 7.5 7.5 7.5 7.6 |
| candidates | | | | | | 7.6 7.6 7.8 7.8 8.0] |

Another example illustrating results obtained using the described Gaussian Process model is described. The example is performed using molecules from the known ChEMBL and GoStar databases. The general approach is to provide a relatively small, initial generation of molecules (i.e., training set), and build ML models based on this training set. Then batch Bayesian optimization in accordance with the described method is performed to select a set of molecules optimizing the activity against a set of targets, from the set of all molecules that contain activity data for the relevant properties. The models are retrained with the new data from the selected set. This process is repeated for a number of cycles or iterations. In this described example, 13403 molecules that contain activity data for at least one of CYP3A4 (UniProt ID P08684) and CYP1A2 (UniProt ID P05177) are extracted from the above-mentioned database. CYP3A4 (cytochrome P450 3A4) is an enzyme in the body, often found in the liver and intestine, and oxidizes toxins so that they can be removed from the body. CYP1A2 (cytochrome P450 1A2) is also an enzyme in the body that localizes to the endoplasmic reticulum. A random initial set of 10 molecules is obtained, and a model for each of the CYPs (i.e., each of the biological properties) is built/trained. Then, 10 rounds or iterations of the Bayesian optimization approach of FIG. 5 is performed, with 20 molecules being selected at each iteration, from the 13393 remaining molecules. After each round, the (known) data for each of the selected molecules is revealed and used to retrain/update the models. Some molecules in the database do not have data for both CYPs, meaning that the models may receive less data in each round or iteration.

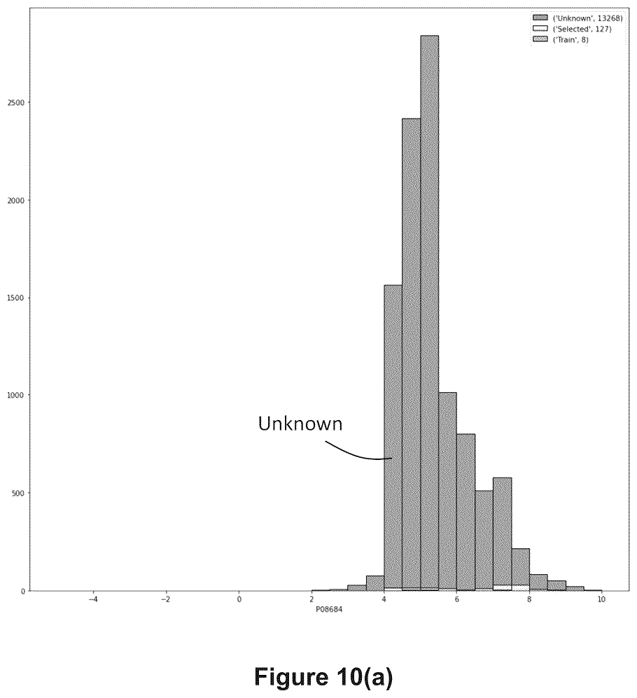

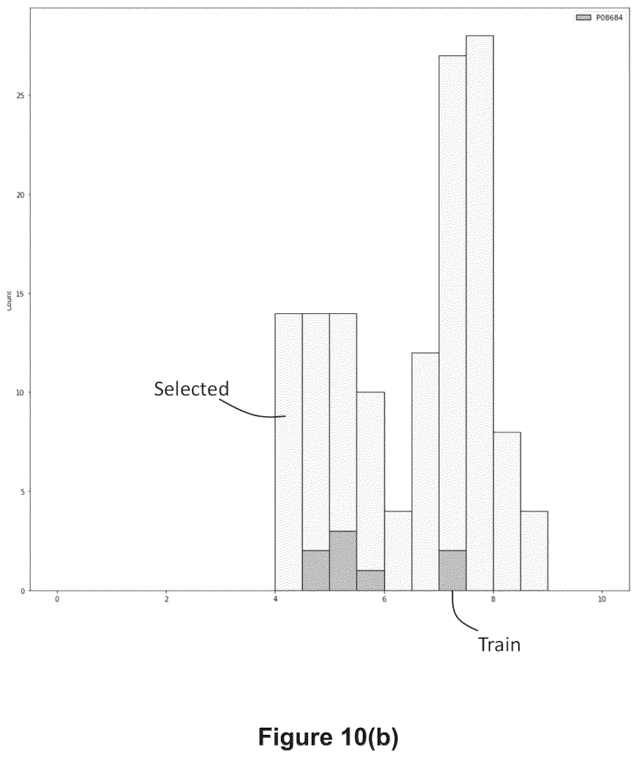

FIG. 10(a) shows a plot illustrating a distribution of CYP3A4 activity values in the set or population of 13403 molecules. In particular, FIG. 10(a) shows the breakdown of these 13403 molecules into 8 molecules in the initial training set, 127 molecules being selected during the iterative optimization, and the remaining or unknown 13268 molecules. As mentioned above, some molecules in the database have known data for only one of the CYPs. In this case, although 10 molecules are selected for the initial training set, only 8 of these have CYP3A4 data. FIG. 10(b) shows a plot illustrating the distribution of CYP3A4 activity values of the molecules in the training and selected sets in FIG. 10(a) and described above, as they can be seen more clearly than in FIG. 10(a).

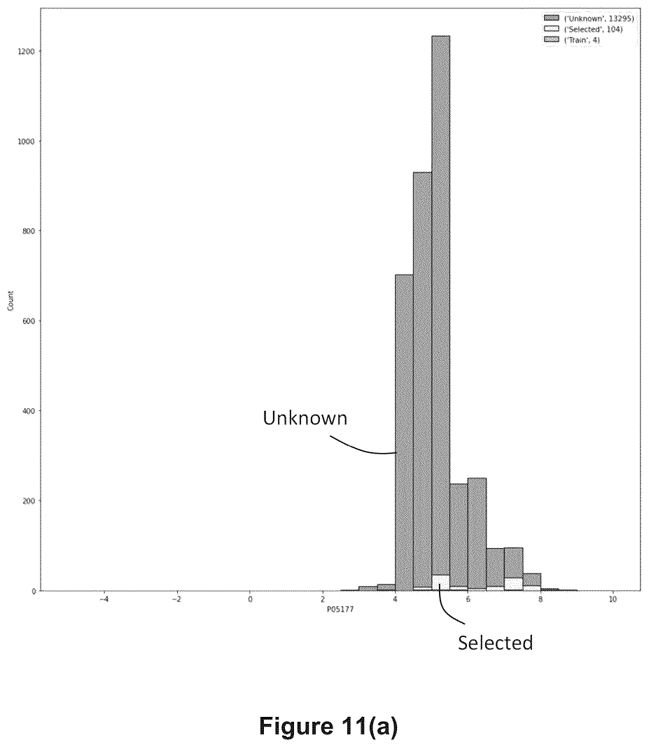

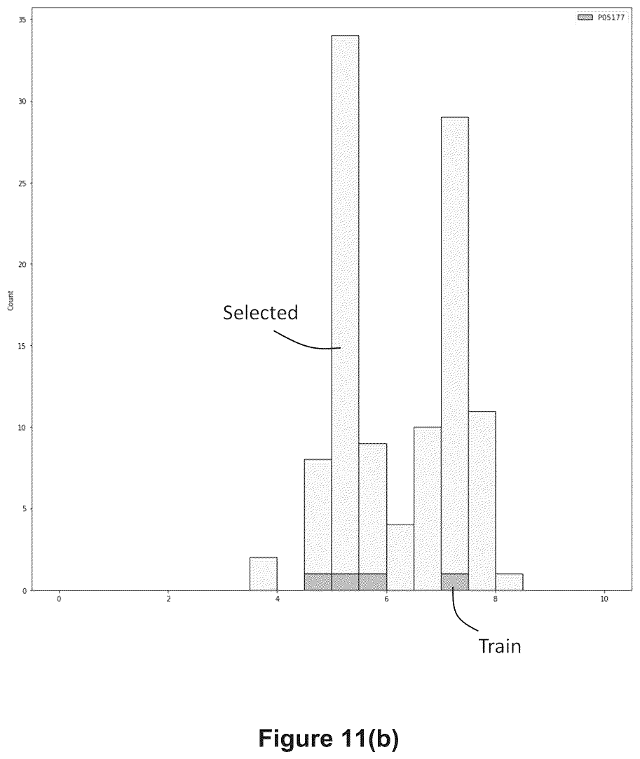

FIGS. 11(a) and 11(b) show corresponding plots to FIGS. 10(a) and 10(b), respectively, but for a distribution of CYP1A2 activity values instead of CYP3A4. In this case, only 4 of the 10 initially selected molecules for training the model have CYP1A2 data available. 104 molecules with available CYP1A2 data were selected across the 30 iterations.

Overall, there is a relatively large enrichment of activity in the selected compounds, both when compared to random selections (analyzing data distributions) and compared to baselines that do not use active learning in accordance with the method described herein. These results are particularly promising given that only 4 and 8 values (from the 10 initial data points) are used, respectively, for the above-outlined targets.

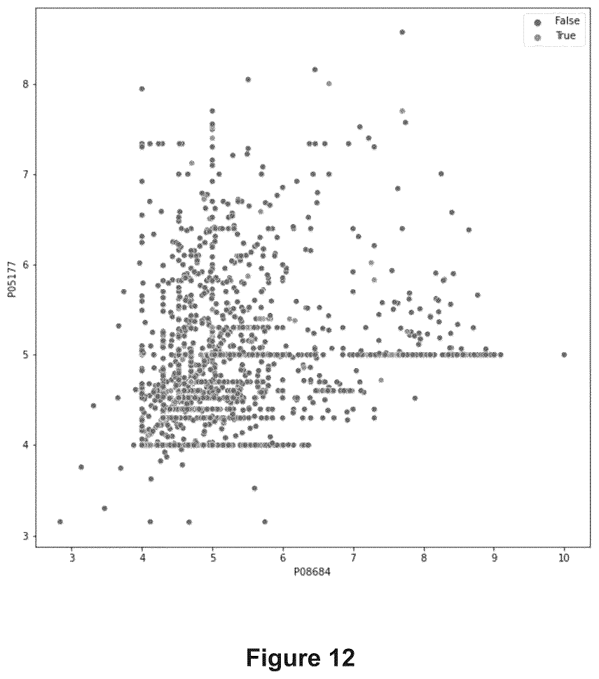

FIG. 12 shows a plot of the CYP3A4 and CYP1A2 activity values for molecules in the set for which both of these values are available, i.e., both are measured in ChEMBL+GoStar. FIG. 12 also indicates which of these molecules were selected when performing the iterations of the described method (‘True’), with the remaining molecules not having been selected (‘False’). With the Pareto frontier being on the upper right hand side of the plot (maximizing activity values), it may be seen that, even though only around 200 molecules out of around 13000 molecules in the population have been selected, close proximity to the Pareto frontier is achieved in the selected set of molecules.

A further example illustrating the described method is provided in terms of free energy perturbation calculations. A dataset of 1921 molecules and corresponding Relative Binding Free Energy (RBFE) calculations are extracted from ‘Reaction-Based Enumeration, Active Learning, and Free Energy Calculations to Rapidly Explore Synthetically Tractable Chemical Space and Optimize Potency of Cylin-Dependent Kinase 2 Inhibitors’, Konze et al., J. Chem. Inf. Model., 2019, 59, 9, 3782-3793. The example starts with the initial training set of 935 molecules from the cited reference, and then 30 rounds or iterations of the method described herein are performed with 10 molecules being selected at each round. The objective is to minimize the RBFE calculation result, measured as ‘Pred dG (kcal/mol)’.

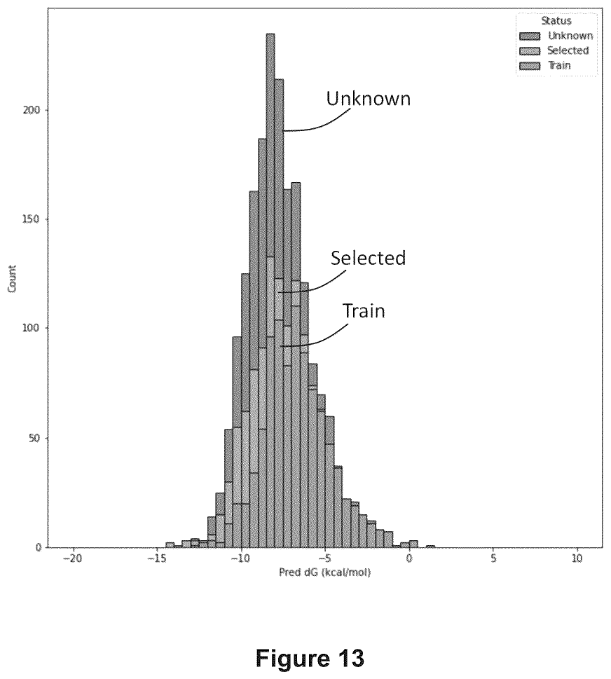

FIG. 13 shows a plot illustrating the distribution of the RBFE values of molecules in the dataset. In particular, FIG. 13 distinguishes between the 935 molecules in the initial training set (‘Train’), the molecules selected when performing the iterations of the described method (‘Selected’), and the remaining molecules in the dataset (‘Unknown’). The lower section of each bar indicates the ‘Train’ molecules, the middle section of each bar indicates the ‘Selected’ molecules, and the upper section of each bar indicates the ‘Unknown’ molecules.

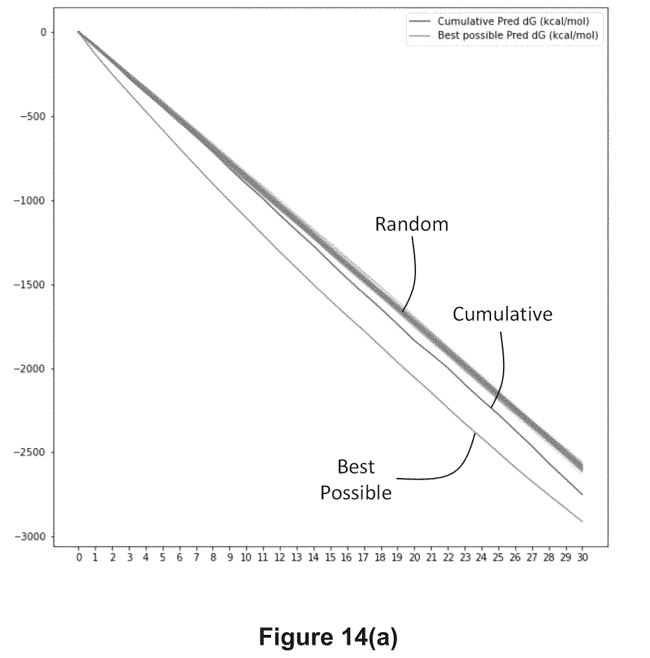

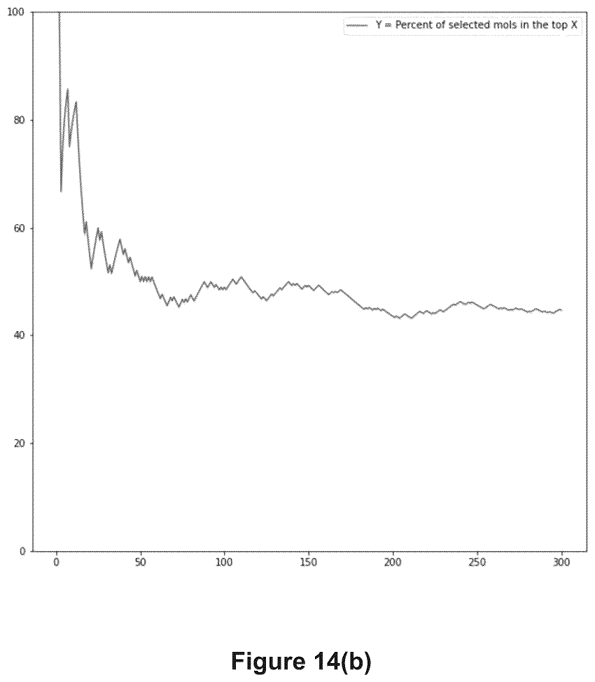

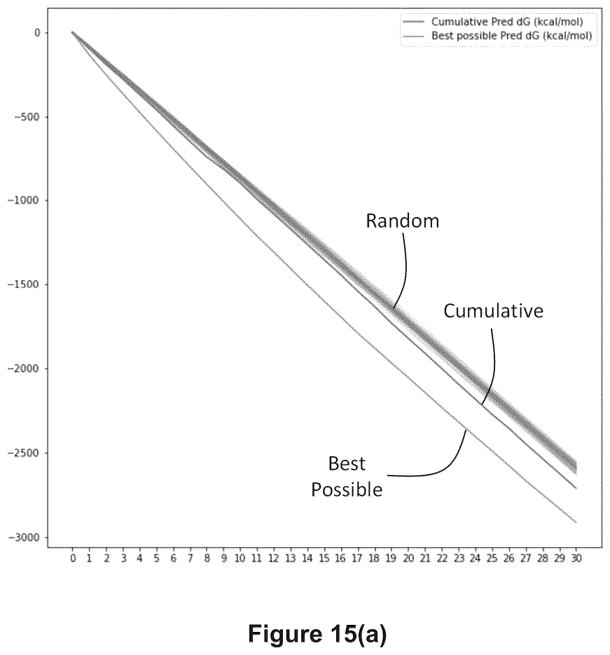

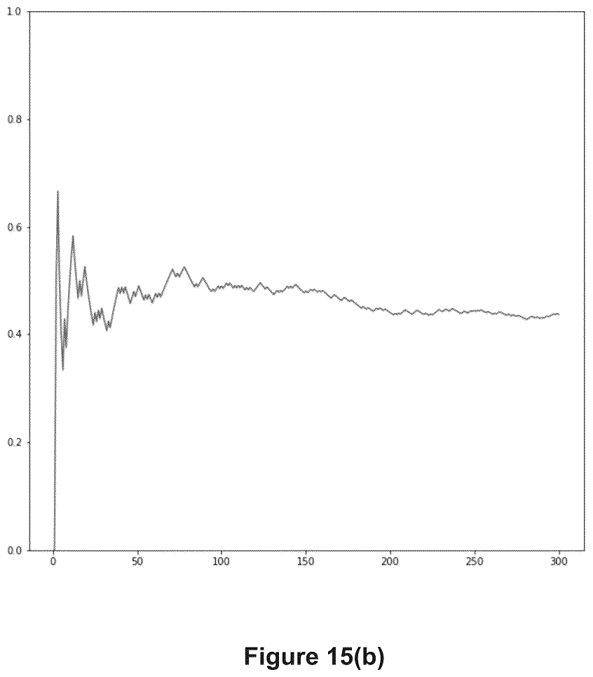

FIG. 14(a) shows a plot of how the cumulative RBFE values under the optimal selection, i.e., by choosing the selected molecules with the lowest dG values, varies with successive iterations of the described method (‘Cumulative Pred dG’). This is compared against optimally selected sets (‘Best possible Pred dG’) and randomly selected sets. FIG. 14(b) then shows a plot of a percentage of the selected molecules in FIG. 14(a) after 30 iterations of the described method that are in the top x of molecules in the dataset according to minimizing the RBFE values. For instance, for x=10, 80% of the lowest dG molecules were found at the end of 30 iterations. At x=1, the 100% result means that the lowest dG molecule was selected. FIG. 15(a) shows the plot of FIG. 14(a), except that FIG. 15(a) shows results of a random forest model greedily selected sets instead of the sets selected via the described method. FIG. 15(b) shows a plot of a percentage of the selected molecules in FIG. 14(a) after 30 iterations of the random forest model that are in the top x of molecules in the test set according to minimizing the RBFE values.

The above examples describe the use of a Gaussian Process model to perform the described Bayesian statistical approach; however, different Bayesian model architectures may be used. For instance, a Bayesian statistical model in the form of a Bayesian neural network, or a deep neural network with dropout providing an uncertainty estimate, may be used in examples of the invention. Furthermore, it will be understood that model ensembles of any generic architecture may be used.

The above examples describe the use of a Bayesian statistical model to select compounds or molecules, from a population, for synthesis, e.g., as part of a drug discovery process. In examples of the invention, compounds or molecules that are selected using the described Bayesian statistical approach may be used for a different purpose. For instance, the described approach may be used to select on which molecules, from a population, to perform molecular dynamics analysis. It may be the case that performing certain physics-based simulations are resource intensive, e.g., they are time consuming and/or require high computer processing capacity, such that computational resources may need to be allocated in a manner that maximizes insights into certain molecular dynamics given the level of computing resource is available.

Many modifications may be made to the above-described examples without departing from the spirit and scope of the invention as defined herein with particular reference to the appended clauses and claims.

### CLAUSES

Clause 1. A method for computational drug design, comprising:

defining a population of a plurality of compounds, each compound having one or more structural features;
defining a training set of compounds from the population for which a plurality of properties are known;
defining a plurality of objectives each defining a desired property;
training, using the training set of compounds, a Bayesian statistical model to output a probability distribution approximating properties of compounds in the population as an objective function of structural features of the compounds in the population;
determining a subset of a plurality of compounds from the population which are not in the training set, the subset being determined according to an optimization of an acquisition function based on the probability distribution from the trained Bayesian statistical model and based on the defined plurality of objectives; and,
selecting at least some of the compounds in the determined subset for synthesis.

Clause 2. The method according to Clause 1, comprising, for one or more of the objectives, mapping a preference associated with the property of the respective objective by applying a respective utility function to the probability distribution from the Bayesian statistical model to obtain a preference-modified probability distribution, wherein optimization of the acquisition function is based on the preference-modified probability distribution.

Clause 3. The method according to Clause 2, wherein the preference is indicative of a priority of the respective objective relative to other ones of the plurality of objectives.

Clause 4. The method according to Clause 2 or Clause 3, wherein, for one of the properties of one of the compounds, the lower an uncertainty value associated with the probability distribution for the property, the greater the preference associated with the respective property.

Clause 5. The method according to any of Clauses 2 to 4, wherein the preference is a user-defined preference.

Clause 6. The method according to any of Clauses 2 to 5, wherein one or more of the utility functions are piecewise functions.

Clause 7. The method according to Clause 6, wherein the piecewise functions are piecewise linear functions.

Clause 8. The method according to any previous clause, wherein optimizing the acquisition function comprises evaluating the acquisition function for each compound in the population, optionally excluding the compounds in the training set, and wherein the subset is determined based on the evaluated acquisition function values.

Clause 9. The method according to any previous clause, wherein the optimization of the acquisition function based on the defined plurality of objectives provides a Pareto-optimal set of compounds, and wherein one or more of the plurality of compounds for the determined subset are selected from the Pareto-optimal set.

Clause 10. The method according to Clause 9, wherein selection from the Pareto-optimal set is according to user-defined preference.

Clause 11. The method according to any previous clause, wherein the probability distribution from the Bayesian statistical model includes a probability distribution for each property associated with each respective one of the plurality of objectives.

Clause 12. The method according to Clause 11, comprising mapping the plurality of probability distributions from the Bayesian statistical model to a one-dimensional aggregated probability distribution by applying an aggregation function to the plurality of probability distributions, wherein optimization of the acquisition function is based on the aggregated probability distribution.

Clause 13. The method according to Clause 12, wherein the aggregation function comprises one or more of: a sum operator; a mean operator; and a product operator.

Clause 14. The method according to any previous clause, wherein the acquisition function is at least one of: an expected improvement function; a probability of improvement function; and a confidence bounds function.

Clause 15. The method according to any of Clauses 1 to 11, wherein the acquisition function is a multi-dimensional acquisition function, wherein each dimension corresponds to a respective objective of the plurality of objectives; optionally wherein the multi-dimensional acquisition function is a hypervolume expected improvement function.

Clause 16. The method according to any previous clause, wherein training the Bayesian statistical model comprises tuning a plurality of hyperparameters of the Bayesian statistical model, wherein tuning the hyperparameters comprises application of a combination of a maximum likelihood estimation technique and a cross validation technique.

Clause 17. The method according to any previous clause, wherein determining the subset of the plurality of compounds comprises:

identifying one compound from the population that is not in the training set by optimizing the acquisition function based on the probability distribution from the trained Bayesian statistical model and based on the defined plurality of objectives, and repeating the steps of:
retraining the Bayesian statistical model using the training set of compounds and the one or more identified compounds; and,
identifying one compound from the population that is not in the training set, and which is not the one or more previously identified compounds, by optimizing the acquisition function based on the probability distribution from the retrained Bayesian statistical model and based on the defined plurality of objectives,
until the plurality of compounds have been identified for the subset.

Clause 18. The method according to Clause 17, wherein retraining the Bayesian statistical model comprises setting one or more fake property values for the one or more identified compounds in the Bayesian statistical model.

Clause 19. The method according to Clause 18, wherein the fake property values are set according to one of: a kriging believer approach; and a constant liar approach.

Clause 20. The method according to any previous clause, wherein in the Bayesian statistical model each compound is represented as a bit vector with bits of the bit vector indicating the presence or absence of respective structural features in the compound.

Clause 21. The method according to any previous clause, wherein the Bayesian statistical model is a Gaussian process model.

Clause 22. The method according to any previous clause, wherein the probability distribution from the trained Bayesian statistical model includes a posterior mean indicative of approximated property values of compounds in the population, and a posterior variance indicative of an uncertainty associated with the approximated property values in the population.

Clause 23. The method according to any previous clause, wherein one or more weighting parameters of the acquisition function are modified in accordance with a desired strategy of a drug design process utilizing the outlined method.

Clause 24. The method according to Clause 23, wherein the desired strategy includes a balance between an exploitation strategy, dependent on a weighting parameter of the acquisition function associated with the posterior mean, and an exploration strategy, dependent on a weighting parameter of the acquisition function associated with the posterior variance.

Clause 25. The method according to Clause 23 or Clause 24, wherein the weighting parameters are user-defined to set the desired strategy.

Clause 26. The method according to any previous clause, wherein the Bayesian statistical model uses a kernel indicative of a similarity between pairs of compounds in the population to approximate the biological properties of the compounds.

Clause 27. The method according to Clause 26, wherein the kernel is a Tanimoto similarity kernel.

Clause 28. The method according to any previous clause, comprising synthesizing at least some of the selected compounds of the determined subset to determine at least one property of the selected compounds.

Clause 29. The method according to Clause 28, comprising adding the synthesized compounds to the training set to obtain an updated training set.

Clause 30. The method according to Clause 29, comprising:

training, using the updated training set of compounds, an updated Bayesian statistical model to output the probability distribution approximating the objective function;
determining a new subset of a plurality of compounds from the population which are not in the updated training set, the new subset being determined according to an optimization of the acquisition function that is dependent on the approximated properties from the updated Bayesian statistical model and on the defined plurality of objectives; and,
selecting at least some of the compounds in the determined new subset for synthesis.

Clause 31. The method according to Clause 30, comprising synthesizing the selected compounds of the determined new subset to determine at least one property of the selected compounds.

Clause 32. The method according to Clause 31, comprising updating the training set by adding the synthesized compounds thereto.

Clause 33. The method according to any of Clauses 29 to 32, comprising iteratively performing the steps of:

training, using the updated training set of compounds, an updated Bayesian statistical model to output the probability distribution approximating the objective function;
determining a new subset of a plurality of compounds from the population which are not in the updated training set, the new subset being determined according to an optimization of the acquisition function that is dependent on the approximated properties from the updated Bayesian statistical model and on the defined plurality of objectives;
selecting at least some of the compounds in the determined new subset for synthesis; synthesizing the selected compounds of the determined subset to determine at least one property of the selected compounds; and,
adding the synthesized compounds to the training set to obtain an updated training set, until a stop condition is satisfied.

Clause 34. The method according to Clause 33, wherein the stop condition includes at least one of: one or more of the synthesized compounds achieve the plurality of objectives; one or more of the synthesized compounds are within acceptable thresholds of the respective plurality of objectives; and a maximum number of iterations have been performed.

Clause 35. The method according to any of Clauses 28 to 34, wherein a synthesized compound that achieves the plurality of objectives, or is within acceptable thresholds of the respective plurality of objectives, is a candidate drug or therapeutic molecule having a desired biological, biochemical, physiological and/or pharmacological activity against a predetermined target molecule.

Clause 36. The method according to Clause 35, wherein the predetermined target molecule is an in vitro and/or in vivo therapeutic, diagnostic or experimental assay target.

Clause 37. The method according to Clause 35 or Clause 36, wherein the candidate drug or therapeutic molecule is for use in medicine; for example, in a method for the treatment of an animal, such as a human or non-human animal.

Clause 38. The method according to any previous clause, wherein each of the objectives is user-defined.

Clause 39. The method according to any previous clause, wherein each of the objectives includes at least one of: a desired value for the respective property; a desired range of values for the respective properties; and a desired value for the respective property to be maximized or minimized.

Clause 40. The method according to any previous clause, wherein a number of compounds in the selected subset is user-defined.

Clause 41. The method according to any previous clause, wherein the structural features of each of the plurality of compounds in the population correspond to fragments, chemical moieties or chemical groups present in the compound.

Clause 42. The method according to Clause 41, wherein the fragments, chemical moieties or chemical groups present in each of the plurality of compounds are represented as a molecular fingerprint; optionally wherein the molecular fingerprint is an Extended Connectivity Fingerprint (ECFP), optionally ECFP0, ECFP2, ECFP4, ECFP6, ECFP8, ECFP10 or ECFP12.

Clause 43. The method according to any previous clause, wherein the properties or at least one property is a biological, biochemical, chemical, biophysical, physiological and/or pharmacological property of each of the compounds.

Clause 44. The method according to any previous clause, wherein the properties include one or more of: activity; selectivity; toxicity; absorption; distribution; metabolism; and excretion.

Clause 45. A compound identified by the method of any previous clause.

Clause 46. A non-transitory, computer-readable storage medium storing instructions thereon that when executed by a computer processor causes the computer processor to perform the method of any of Clauses 1 to 44.

Clause 47. A computing device for computational drug design, comprising:

an input arranged to receive data indicative of a population of a plurality of compounds, each compound having one or more structural features, to receive data indicative of a training set of compounds from the population for which a plurality of properties are known, and to receive data indicative of a plurality of objectives each defining a desired property;
a processor arranged to train, using the training set of compounds, a Bayesian statistical model to provide a probability distribution approximating properties of compounds in the population as an objective function of structural features of the compounds in the population, and arranged to determine a subset of a plurality of compounds from the population which are not in the training set, the subset being determined according to an optimization of an acquisition function based on the probability distribution from the trained Bayesian statistical model and based on the defined plurality of objectives; and,
an output arranged to output the determined subset; optionally wherein the computing device is arranged to select at least some of the compounds in the determined subset for synthesis.

Clause 48. The computing device according to Clause 47, wherein the processor is configured to read computer-readable code to execute at least some of the steps of a method according to any of Clauses 1 to 44.

Clause 49. A method for computational drug design, comprising:

defining a population of a plurality of compounds, each compound having one or more structural features;
defining a training set of compounds from the population for which a plurality of properties are known;
defining a plurality of objectives each defining a desired property;
training, using the training set of compounds, a Bayesian statistical model to output a probability distribution approximating properties of compounds in the population as an objective function of structural features of the compounds in the population;
determining a subset of a plurality of compounds from the population which are not in the training set, the subset being determined according to an optimization of an acquisition function based on the probability distribution from the trained Bayesian statistical model and based on the defined plurality of objectives; and,
selecting at least some of the compounds in the determined subset for performing molecular dynamics analysis.

Clause 50. The method according to Clause 49, comprising performing the molecular dynamics analysis based on the selected compounds.

What is claimed is:

1. A method for computational drug design, comprising:
defining a population of a plurality of compounds, each compound having one or more structural features;
defining, from the population, a training set of compounds for which a plurality of properties are known;
defining a plurality of objectives, each objective defining a respective desired property;
training, using the training set of compounds, a Bayesian statistical model to output a probability distribution approximating properties of compounds in the population as an objective function of structural features of the compounds in the population;
determining, from the population, a subset of the plurality of compounds that are not in the training set, the subset being determined according to an optimization of an acquisition function based on (i) the probability distribution from the trained Bayesian statistical model and (ii) the defined plurality of objectives; and,
selecting at least some of the compounds in the determined subset for synthesis.

2. The method according to claim 1, further comprising:
for one or more of the plurality of objectives, mapping a preference associated with a property of the respective objective by applying a respective utility function to the probability distribution from the trained Bayesian statistical model to obtain a preference-modified probability distribution, wherein the optimization of the acquisition function is based on the preference-modified probability distribution.

3. The method according to claim 2, wherein the preference is indicative of a priority of the respective objective relative to other objectives of the plurality of objectives.

4. The method according to claim 2, wherein:
the plurality of compounds includes a first compound having a first property, the first property including a first probability distribution; and
the first property is associated with a greater preference when an uncertainty value associated with the first probability distribution decreases.

5. The method according to claim 1, wherein optimizing the acquisition function includes:
for each compound of the plurality of compounds in the population:
evaluating the acquisition function for the respective compound to determine a respective acquisition function value, wherein the subset of the plurality of compounds is determined based on a plurality of acquisition function values corresponding to the plurality of compounds in the population.

6. The method according to claim 1, wherein:
the optimization of the acquisition function based on the defined plurality of objectives provides a Pareto-optimal set of compounds; and
the determined subset of the plurality of compounds includes one or more compounds that are selected from the Pareto-optimal set.

7. The method according to claim 1, wherein:
the probability distribution from the Bayesian statistical model includes a plurality of first probability distributions, each of the first probability distributions corresponding to a respective property associated with a respective objective of the plurality of objectives.

8. The method according to claim 7, further comprising mapping the plurality of first probability distributions from the Bayesian statistical model to a one-dimensional aggregated probability distribution by applying an aggregation function to the plurality of first probability distributions, wherein the optimization of the acquisition function is based on the aggregated probability distribution.

9. The method according to claim 1, wherein:
the acquisition function has multiple dimensions, each dimension corresponding to a respective objective of the plurality of objectives.

10. The method according to claim 1, wherein:
training the Bayesian statistical model comprises tuning a plurality of hyperparameters of the Bayesian statistical model, the tuning including applying a combination of a maximum likelihood estimation technique and a cross validation technique.

11. The method according to claim 1, wherein determining the subset of the plurality of compounds comprises:
identifying one compound from the population that is not in the training set by optimizing the acquisition function based on the probability distribution from the trained Bayesian statistical model and based on the defined plurality of objectives, and repeating the steps of:
retraining the Bayesian statistical model using the training set of compounds and the one or more identified compounds; and,
identifying one compound from the population that is not in the training set and not the one or more previously identified compounds, by optimizing the acquisition function based on the probability distribution from the retrained Bayesian statistical model and based on the defined plurality of objectives,
until the plurality of compounds have been identified for the subset.

12. The method according to claim 11, wherein retraining the Bayesian statistical model comprises setting one or more fake property values for the one or more identified compounds in the Bayesian statistical model, wherein the fake property values are set according to one of: a kriging believer approach and a constant liar approach.

13. The method according to claim 1, wherein the Bayesian statistical model is a Gaussian process model.

14. The method according to claim 1, wherein:
one or more weighting parameters of the acquisition function are modified in accordance with a desired strategy of a drug design process, the desired strategy including an optimization of:
(i) an exploitation strategy, dependent on a weighting parameter of the acquisition function associated with the posterior mean; and
(ii) an exploration strategy, dependent on a weighting parameter of the acquisition function associated with the posterior variance.

15. The method according to claim 1, comprising synthesizing at least some of the selected compounds of the determined subset to determine at least one property of the selected compounds, and adding the synthesized compounds to the training set to obtain an updated training set.

16. The method according to claim 15, comprising:
training, using the updated training set of compounds, an updated Bayesian statistical model to output the probability distribution approximating the objective function;
determining a new subset of a plurality of compounds from the population which are not in the updated training set, the new subset being determined according to an optimization of the acquisition function that is dependent on the approximated properties from the updated Bayesian statistical model and on the defined plurality of objectives; and,
selecting at least some of the compounds in the determined new subset for synthesis.

17. The method according to claim 16, comprising synthesizing the selected compounds of the determined new subset to determine at least one property of the selected compounds, and updating the training set by adding the synthesized compounds thereto.

18. The method according to claim 17, comprising iteratively performing the steps of:
training, using the updated training set of compounds, an updated Bayesian statistical model to output the probability distribution approximating the objective function;
determining a new subset of a plurality of compounds from the population which are not in the updated training set, the new subset being determined according to an optimization of the acquisition function that is dependent on the approximated biological properties from the updated Bayesian statistical model and on the defined plurality of objectives;
selecting at least some of the compounds in the determined new subset for synthesis;
synthesizing the selected compounds of the determined subset to determine at least one property of the selected compounds; and,
adding the synthesized compounds to the training set to obtain an updated training set,
until a stop condition is satisfied.

19. A non-transitory, computer-readable storage medium storing instructions that, when executed by a computer system having one or more processors and memory, cause the computer system to perform operations comprising:
receiving (i) data indicative of a population of a plurality of compounds, each compound having one or more structural features, (ii) data indicative of a training set of compounds from the population for which a plurality of biological properties are known, (iii) data indicative of a plurality of objectives each defining a desired biological property;
training, using the training set of compounds, a Bayesian statistical model to output a probability distribution approximating properties of compounds in the population as an objective function of structural features of the compounds in the population;
determining, automatically and without user intervention, a subset of a plurality of compounds from the population which are not in the training set, the subset being determined according to an optimization of an acquisition function based on the probability distribution from the trained Bayesian statistical model and based on the defined plurality of objectives; and,
causing output of the determined subset of the plurality of compounds, wherein at least a portion of the compounds in the determined subset are selected for synthesis.

20. A computing device for computational drug design, comprising:
one or more processors; and
memory coupled to the one or more processors, the memory storing one or more instructions configured to be executed by the one or more processors, the one or more instructions including instructions for:
receiving (i) data indicative of a population of a plurality of compounds, each compound having one or more structural features, (ii) data indicative of a training set of compounds from the population for which a plurality of biological properties are known, (iii) data indicative of a plurality of objectives each defining a desired biological property;
training, using the training set of compounds, a Bayesian statistical model to output a probability distribution approximating properties of compounds in the population as an objective function of structural features of the compounds in the population;
determining, automatically and without user intervention, a subset of a plurality of compounds from the population which are not in the training set, the subset being determined according to an optimization of an acquisition function based on the probability distribution from the trained Bayesian statistical model and based on the defined plurality of objectives; and
causing output of the determined subset of the plurality of compounds, wherein at least a portion of the compounds in the determined subset are selected for synthesis.
